SEQUENCE LISTING (8 sequences)

SEQ ID NO: 1 - DNA, 34 bp, Artificial sequence
  Probe of capture
  misc_binding (1)..(1): Spacer including one amine group and one carbon chain with 6 carbons
ttttttttttttggacaaaatacctgtattcctc

SEQ ID NO: 2 - DNA, 37 bp, Artificial sequence
  Probe of capture
  misc_binding (1)..(1): Spacer including one amine group and one carbon chain with 6 carbons
ttttttttttttaatagcactgggagcattgaggctc

SEQ ID NO: 3 - DNA, 30 bp, Artificial sequence
  Synthetic Oligonucleotide
  misc_binding (1)..(1): Spacer including one amine group and one carbon chain with 6 carbons
  modified_base (30)..(30): /mod_base= Thymin biotin fixed on T
tttttttttttttttttttttttttttttt

SEQ ID NO: 4 - DNA, 22 bp, Artificial sequence
  Probe of capture
  modified_base (1)..(1): /mod_base= Guanin fluorescein fixed on G
gaggaatacaggtattttgtcc

SEQ ID NO: 5 - DNA, 25 bp, Artificial sequence
  Probe of capture
  modified_base (1)..(1): /mod_base= Guanine fluorescein fixed on G
gagcctcaatgctcccagtgctatt

SEQ ID NO: 6 - DNA, 34 bp, Artificial sequence
  Probe of capture
  misc_binding (1)..(1): Spacer including one amine group and one carbon chain with 6 carbons
ttttttttttttggacaaaatacctgtattcctt

SEQ ID NO: 7 - DNA, 21 bp, Artificial sequence
  Probe of capture
  modified_base (1)..(1): /mod_base= Guanin Phosphate group on G“
  modified_base (21)..(21): /mod_base= Cytidin fluorescein fixed on C
gcctgtccagggatctgctcc

SEQ ID NO: 8 - DNA, 39 bp, Artificial sequence
  Probe of revelation
  modified_base (1)..(1): /mod_base= Guanin phosphate group on G”
  modified_base (39)..(39): /mod_base= Thymin biotin fixed on T“
gcctgtccagggatctgctcctttttttttttttttttt

(19) United States
(12) Patent Application Publication
(10) Pub. No.: US 2006/0292552 A1
(43) Pub. Date: Dec. 28, 2006

(54) Method for the detection and multiplex quantification of analytes in a sample, using microspheres

(75) Inventors: Guillaume Haquette, Vitrolles (FR); Philippe Poncelet, Marseille (FR); Maxime Moulard, Auriol (FR); Michel Canton, Levallois Perret (FR)

Correspondence Address: FOLEY AND LARDNER LLP;SUITE 500, 3000 K STREET NW, WASHINGTON, DC, 20007, US

(73) Assignee: BIOCYTEX

(21) Appl. No.: 10/558,082

(22) PCT Filed: May 26, 2004
(86) PCT No.: PCT/FR04/01307

§ 371 Date: Apr. 18, 2006

(30) Foreign Application Priority Data
May 26, 2003 (FR) 0306354

(51) Int. Cl. C12Q 1/70; C12Q 1/68; G01N 33/53; G01N 33/551

(52) U.S. Cl. 435/5; 435/6; 435/7.5; 436/524

(57) ABSTRACT

The invention relates to a method for the detection and multiplex quantification of analytes in a sample, using functionalised microspheres, whereby said microspheres are magnetised after the sample has been brought into contact therewith. The inventive method is particularly suitable for the detection and multiplex quantification of several analytes by means of flow cytometry. The invention also relates to a kit which is used for the detection and/or quantification of several analytes in order to carry out the inventive method, comprising a suspension of functionalised non-magnetic microspheres, a ferrofluid solution and a solution with at least one conjugate.

The present invention relates to a method for the detection and/or multiplex quantification of analytes in a sample using functionalized microspheres, these microspheres being magnetized after the step of bringing the sample into contact with these microspheres. The method of the invention is particularly suitable for the detection and multiplex quantification of several analytes by flow cytometry. The invention also relates to a kit for the detection and/or quantification of several analytes in order to carry out the method according to the invention, which comprises a suspension of functionalized non-magnetic microspheres, a solution of ferrofluids and a solution of at least one conjugate.

The development and diversification of in vitro diagnosis increasingly require the setting up of means for the rapid detection and identification of various compounds and microorganisms. This need relates at the same time to the human or animal health sector, for example for the search for or the assaying of specific antigens or pathogenic agents, the agrofoods sector, for instance quality control and the screening for possible contaminants in products intended for food, or the environmental sector when it involves, for example, preventing any biological risk or detecting impurities, pesticides or various polluting agents.

The use of immunoassays on microbeads combined with analytical systems of flow cytometry type constitutes one of the technological pathways most suited to these needs, and many approaches based on this principle have been proposed in the state of the art.

For example, the international patent applications published under the numbers WO 98/51435, WO 94/09368 and WO 90/15666 and the European patent application published under the number EP 180384 describe magnetic or fluorescent particles of specific type that can be used in diagnostic methods.

Other documents of the prior art propose various protocols for the identification and/or assaying of multiple analytes on microbeads. Thus, by way of example, the international application published under the number WO 90/05305 concerns a method and its corresponding kit for detecting and/or assaying several analytes in a sample by means of an agglutination method using several subpopulations of fluorescent beads. The fluorescence of the aggregates formed can be measured by flow cytometry, image analysis or a laser scanning system.

The American patent granted under the number U.S. Pat. No. 4,665,020 concerns a method for assaying an antigen by flow cytometry using two populations of spheres of different diameter, the largest being coated with an antibody specific for the antigen, and the smallest being fluorescent. The assay is carried out according to the principle of a sandwich method or a competition method according to the ligand attached to the fluorescent spheres (antibody or antigen).

The international application published under the number WO 97/14028 concerns a method of analysis by flow cytometry for the detection of several analytes of interest, in which use is made of a plurality of subpopulations of beads for which at least one of the classification parameters for the analysis by flow cytometry differs from one subpopulation to the other. Each subpopulation is coupled to a compound that reacts specifically with one of the analytes to be assayed. The subpopulation of beads, and therefore the nature of the compound that has reacted with the corresponding analyte, is identified by cytometry, by means of the analysis of all the classification parameters of each subpopulation.

The international application published under the number WO 98/20351 concerns a method for determining the presence of one or more analytes in a sample, in which use is made of “test” populations of microparticles, each of the populations carrying a ligand specific for an analyte, and reference microparticles that do not react with any of the analytes being investigated. The assaying is carried out by counting the number of free microparticles in each “test” population and comparing it with that of the reference microparticles. The counting is carried out according to various methods, including preferably cytometry.

The international application published under the number WO 96/31777 is directed toward a method for the detection of microorganisms in a sample using at least one type of detectable particle carrying a ligand specific for the microorganisms being investigated. The microorganisms attached to the particles are then revealed using a second ligand carrying a fluorescent marker.

The American patent granted under the number U.S. Pat. No. 6,280,618 concerns a method for individually detecting a plurality of analytes in which use is made of a mixture of populations of magnetic microparticles that can be differentiated from one another and that each carry a different ligand. The microparticles of each group are separated from the medium and then suspended in a second liquid medium in which they are analyzed by flow cytometry.

The international application published under the number WO 93/02260 concerns a method of flow cytometry for simultaneously detecting several analytes in the same sample, and the reagent for the implementation thereof. This reagent consists of a mixture of several subpopulations of microspheres, each subpopulation carrying at the surface a specific ligand capable of forming a specific binding pair with one of the analytes being investigated. The detection of the analytes attached to the microspheres is carried out after addition of an agent carrying a fluorochrome, capable of binding to the binding pairs formed.

These methods for the simultaneous detection of several analytes in the same sample can also comprise one or more magnetic separation steps.

In fact, such a magnetic separation step makes it possible to facilitate the assays in certain complex media where the antigens of interest must be specifically isolated (it being possible for a cytometric analysis to prove impossible to carry out in the presence of certain microparticles). This is the case, for example, for analyses in agrofoods, paper-making and wastewater treatment, where molecules/particles present in various liquefied ground materials (pulps, musts, dairy products or even cheeses, fruit juices, ground vegetable materials, fermentation liquors, etc.) are investigated, which preparations cannot be filtered because of the risk of losing the analyte to be assayed.

This is also the case for analyses in environmental and human health sciences, where the particulate content of a large volume of air is concentrated in a liquid by means of a biosampler. In this case also, it is imperative to remove all the particles, dusts, microfibers, pollen, etc., in suspension in the air, the size of which i) either covers that of the trapping beads (1 to 30 μm) and makes it difficult or even impossible to identify them by only light scattering parameters, ii) or makes the analysis incompatible by blocking the cytometer (high risk from 100 μm), and which are found concentrated in the liquid after biocollection.

In addition, oily (greasy) particles which are incompatible with the correct functioning of the fluidics of a cytometer must also be eliminated. In addition, the use of magnetic separation also makes it possible to rapidly concentrate the agents to be assayed and prevents having to use centrifugation steps for washing the trapping beads.

Such a combination between a specific trapping step on microbeads and a magnetic separation step has already been described in the state of the art, in particular in the patent granted under the number U.S. Pat. No. 6,280,618.

However, the use of magnetic microspheres in a method for the identification and assaying of analytes contained in a sample can present various industrial constraints, and impair the handling and the homogeneity of sampling of the suspensions. This is because magnetic microspheres of different sizes (and often having different contents of magnetizable material) can have different behaviors during the magnetic separation, i.e. longer or shorter magnetization times. Separation yields that are variable according to the families of microbeads present result therefrom, hence a heterogeneity of the results or a prolonging of the magnetic separation phases. In order to overcome this problem, it is necessary to have magnetic microspheres whose content of magnetizable material is adjusted according to size. This of course creates industrial constraints in terms of manufacture or supply.

In addition, magnetic microspheres are dense, which poses practical problems of sedimentation during storage, resuspension and rapid sedimentation during analysis. In fact, the density of magnetic beads is commonly greater than 1.15 and, according to the amount of magnetite, oscillates between 1.15 and 1.50, leading to very rapid sedimentation rates, in particular for particles of large diameter (>2 μm) (cf. European patent application published under the number EP 1248110).

Thus, there exists today a need to develop a novel specific method for the identification and assaying of several analytes contained in a liquid sample, and which comprises one or more magnetic separation steps, said method being capable of circumventing such drawbacks associated with the use of magnetic microspheres.

The subject of American patent U.S. Pat. No. 5,998,224 (published on Dec. 7, 1999, applicant: Abbott Laboratories) is a method for determining the presence or the amount of an analyte in a test sample.

According to a first embodiment, this method comprises bringing the test sample into contact with a mobile solid phase and a magnetic reagent so as to form a reaction mixture in which said analyte binds to said mobile solid phase and said magnetic reagent so as to form a complex, and then applying a magnetic field.

According to a second embodiment, the method comprises bringing the analyte into contact with the magnetic reagent so as to form a first complex, and bringing the magnetic reagent into contact with the mobile solid phase so as to form a second complex, and then applying the magnetic field.

According to an alternative of this second embodiment, the analyte binds to the mobile phase so as to form a first complex and the magnetic reagent binds to the mobile phase so as to form a second complex, and then a magnetic field is applied.

The subject of the American patent application published on Dec. 27, 2001, under the number US 2001/0054580 (Bio-Rad Laboratories, Inc, related to EP 1248110), is a multiplex test for differentiating several analytes in a sample. This test uses magnetic particles as a solid phase and engenders an individual result for each analyte. The magnetic particles can be distinguished from one another via characteristics that make it possible to differentiate them in groups, each group carrying a reagent bound to the surface of the particle, which is different from the reagents present on the particles of the other groups.

In this multiplex test, the sample containing said analytes is brought into contact with magnetic particles.

The subject of the European patent application published on Aug. 5, 1987, under the number EP 230 768 (Syntex Inc), is a method for separating a substance from a liquid medium. This method uses magnetic or non-magnetic particles.

The non-magnetic particles can be functionalized so as to bind to a member of a specific binding pair or to a magnetic particle.

A ferrofluid is described as a magnetic fluid, in which the particles in suspension are ferromagnetic particles. The colloidal magnetic particles of the magnetic fluid can be coated with protein material such as ferric proteins: albumin, gamma globulin, etc. The coating of the magnetic particles with proteins can be accomplished by physical binding, for example absorption, or chemical bonding.

A subject of the present invention is a method for the detection and/or multiplex quantification of analytes that may be contained in a sample, using functionalized non-magnetic microspheres, it being possible, where appropriate, for said analytes to be labeled beforehand with a label, said method being characterized in that it comprises the following steps:
a) bringing said sample into contact with a suspension of functionalized non-magnetic microsphere populations, said microspheres carrying at their surface:
   for all the microsphere populations, a compound A forming a first member of a binding pair, said compound A also being characterized in that it cannot bind with said analytes, and
   for each one of the microsphere populations, a compound B, that is different for each population, capable of forming a specific binding pair with one of said analytes of the sample,
b) adding to the reaction medium obtained in step a) a ferrofluid, which ferrofluid contains magnetic particles which carry at their surface a second binding member capable of forming a specific binding pair with the compound A,
c) at least one step consisting in washing by magnetic separation of the microspheres magnetized in step b),
d) where appropriate, when said analytes are not labeled beforehand, bringing the suspension of magnetized microspheres obtained in step c) into contact with a solution of at least one conjugate, said conjugate comprising a compound C capable of recognizing and of binding specifically with one of said analytes and a label, this step d) preferably being followed by at least one step consisting in washing the microspheres by magnetic separation, and
e) detecting and/or quantifying said label at the surface of the microspheres.

The expression “method for the detection and/or multiplex quantification” is intended to denote in the present description a method for the detection and/or quantification of several analytes of interest in a single test.

The term “sample” is intended to denote in the method according to the present invention any sample that can contain several analytes that it is desired to detect and/or quantify in said sample.

Among the samples that may contain said analytes according to the present invention, mention may in particular be made of biological samples (in particular whole blood, plasma or serum, cerebrospinal fluid, mucous membranes, etc.) or chemical samples derived from all types of samples taken, in particular in the human or animal health, agrofoods or environmental sectors, or else derived from the chemical industry, which samples are taken in order to detect and/or quantify several analytes of interest that may be contained in said sample taken.

The term “analyte” is intended to denote in the present description any compound of interest liable to be able to be detected and/or quantified according to the present invention.

Preferably, said analytes are of protein and derivative type, or are nucleic acids.

The term “protein”, “polypeptide” or “peptide” is used without distinction in the present description to denote a sequence of amino acids or, for their derivatives, containing a sequence of amino acids.

The term “nucleic acid”, “nucleic acid sequence”, “polynucleotide”, “oligonucleotide”, “polynucleotide sequence” or “nucleotide sequence”, which terms will be used without distinction in the present description, is intended to denote a precise chain of nucleotides, which may or may not be modified, making it possible to define a fragment or a region of a nucleic acid, possibly comprising unnatural nucleotides, and which may correspond equally to a double-stranded DNA, a single-stranded DNA and products of transcription of said DNAs. These nucleic acids are isolated from their natural environment, and are natural or artificial.

According to a particular embodiment, such analytes are chemical or biochemical organic compounds that may either be present in solution in a liquid, or present at the surface of a cell or of a particle in suspension in the sample.

As an example of a cell or of a particle that may carry said analyte at its surface, mention may be made, but without being limited thereto, of eukaryotic cells such as a mammalian cell or a yeast, prokaryotic cells such as, for example, a bacterium, and particles such as, for example, a viral particle, a spore or a pollen grain, or any microorganism, in particular a fungus.

A first embodiment of the invention concerns the identification and/or the multiplex assaying, in a sample, of biological agents such as antigens bound to supports, for instance surface antigens of microorganisms, receptors and other membrane structures, or analytes in the free state in a sample, such as proteins, enzymes, metabolites, and other secretion products.

As an example of analytes of interest, mention may in particular be made, but without being limited thereto, of proteins and their derivatives such as glycoproteins or lipoproteins, nucleic acids, carbohydrates, compounds that are lipid in nature, and all natural compounds or compounds that can be obtained by chemical synthesis. Mention may also be made, as examples of analytes, of compounds that exhibit a functional particularity, such as cytokines, cell receptors, antibodies, antigens, toxins, allergens, drugs, pesticides or herbicides, or any pollutant, analytes that it is desired to detect and/or quantify in a sample, whether they are present in solution or, where appropriate, carried at the surface of a cell or of a particle.

Particularly preferably, said compounds are protein toxins, or else -said cell or particle is a microorganism, such as a bacterium or a virus.

When the analytes are present at the surface of a cell or of a particle, the detection and/or quantification of said analytes allows the detection and/or quantification of said cells or particles if said analytes selected are specific for these said cells or particles.

As another example of analytes of interest that may be contained in a sample, mention may be made of compounds present in the atmosphere, naturally or accidentally, which can be collected in a liquid, for instance by means of a biosampler, which samples the particles present in the atmosphere and impacts them into a liquid, generally a buffer such as PBS, or an oil.

A typical example of a biosampler is described in the commercial documentation of the BioSampler® from SKC Inc., Pa., and also in patents U.S. Pat. No. 5,902,385 and U.S. Pat. No. 5,904,752 and in technical publications such as: Buttner et al.: Sampling and analysis of airborne microorganisms, in Manual of environmental Microbiology, ASM Press, Wash. D.C., 1997, pp. 629-640, Improved aerosol collection by combined impaction and centrifugal motion. Willeke K. et al., Aerosol Sci. Tech., 28:439-456 (1998) U.S. Pat. No. 6,468,330 Irving et al.

The expression “analytes labeled beforehand with a label” is intended to mean in the method according to the invention any analyte that it is intended to detect and/or quantify in a sample and that is in labeled form before said method is carried out.

As an example of analytes labeled beforehand mention may be made, but without being limited thereto, of nucleic acids, or PCR products (amplicons), that result from an enzymatic amplification, such as PCR (polymerase chain reaction), which can be obtained in a labeled form, in particular by means of fluorescent labels, these nucleic acid-labeling techniques being well known to those skilled in the art.

The expression “functionalized non-magnetic microspheres” is intended to denote in the method according to the present invention non-magnetic microspheres carrying at their surface a compound A and a compound B.

The compounds A and B can be attached to the non-magnetic microspheres according to methods known to those skilled in the art, such as coupling by covalent bonding, by affinity, or by passive or forced adsorption. Such methods are also used for attaching the compound to the surface of the magnetic particles of the ferrofluid. Such methods for functionalizing various supports have been widely described in the literature, for example in the American patents granted under the numbers U.S. Pat. No. 4,181,636—U.S. Pat. No. 4,264,766—U.S. Pat. No. 4,419,444—U.S. Pat. No. 4,775,619, etc., and in Legastelois S. et al., Latex and diagnostics. 1996, or Le Technoscope Biofutur, 161:1-11; Duke Scientific Corp. Catalog, Palo-Alto, Calif. Technical Note-013A “Reagent Microspheres-Surface properties and Conjugation Methods”.

In the case of coupling by covalent bonding, the microspheres used carry chemical groups capable of reacting with another chemical group carried by the compound A or B so as to form a covalent bond.

As an example of chemical groups that may be present at the surface of the microspheres, mention may be made, but without being limited thereto, of carboxyl, amino, aldehyde and epoxy groups. In the specific case where the analytes that are intended to be characterized are reactive chemical species, one of the chemical groups carried by the non-magnetic microspheres may be capable of reacting specifically with the reactive chemical species of said analytes that it is intended to detect and/or quantify, said chemical group thus also performing the role of the compound B.

To functionalize the microspheres, use may also be made of interaction by affinity, which is generally implemented by two partners of a high affinity binding couple, such as in particular, but without being limited thereto, the couples (poly)carbohydrate/lectin, biotin or biotinylated compounds/avidin or streptavidin, receptor/specific ligand, antigen or hapten/antibody, etc.

The functionalization of the microspheres can also be carried out either directly, or using spacer arms also referred to by the terms “linker” or “spacer”. Functionalization by passive or forced adsorption is known to those skilled in the art, and has already been described in the American patents mentioned above. For the functionalization by passive adsorption, BSA-biotin (bovine serum albumin) (Sigma, Lyons, FR—Ref. A-8549) may, for example, be used.

The functionalized non-magnetic microspheres in the present description may consist of any type of material provided that the latter contains no magnetic constituent. A material that is inert with respect to the analytes of the sample and with respect to the other analytical reagents, that is insoluble in the sample and in all the other reaction media used in the method according to the invention, and that can be functionalized, will preferably be chosen. It may, for example, be a polymer, or a copolymer, or else made of latex, glass or silica.

Provided that these microspheres are not magnetic, there is no particular constraint as regards the choice of the material from which they can be manufactured. Thus, any type of microsphere made of a very broad range of non-magnetic latex can be used. It is also possible to use microspheres made of other materials, more or less suitable according to the analytes to be detected and/or quantified, which are sold in various sizes in non-magnetic form. Thus, the method according to the present invention may, for example, use glass or silica microbeads, materials that are respectively preferably used for trapping blood platelets and nucleic acids (see in particular the international application published under the number WO 94/19600, which describes the use of glass microbeads for trapping (and in this specific case eliminating) activated aggregated platelets).

As examples of polymers or of copolymers, mention may be made, but without being limited thereto, of divinylbenzene, polystyrene, polyvinylpyridine, styrene-butadiene copolymers, acrylonitrile-butadiene copolymers, polyesters, vinyl ester acrylate-acetate copolymers, vinyl ester acrylate-chloride copolymers, polyethers, polyolefins, polyalkylene oxides, polyamides, polyurethanes, polysaccharides, celluloses and polyisoprenes. Crosslinking is useful for many polymers in order to give structural integrity and rigidity to said microspheres.

Thus, according to a particular embodiment of the invention, the method is characterized in that the microspheres are made of a material chosen from the group consisting of latex, a polymer, a copolymer, glass or silica.

The non-magnetic microspheres made of a polymer or copolymer, of latex, of glass or of silica are well known to those skilled in the art and are commercially available, for instance the microsphere ranges provided by the companies Spherotech (Libertyville, US), Polysciences (Warrington, US), Merck Eurolab SA (Fontenay-sous-Bois, FR) for the Estapor range, Duke Scientific (Palo Alto, US), Seradyn (Indianapolis, US), Dynal Biotech for Dyno Particles (Oslo, NO), etc. Other microsphere ranges can be provided by Bang's Labs (Fishers, US) and Polymer Laboratories Ltd (Church Stretton, UK).

Similarly, procedures for adsorption/desorption of nucleic acids onto/from silica beads are described in TechNote #302, Bang's Labs (Fishers, US) for the trapping of nucleic acids.

Moreover, because of the scope of choice of the material of the microspheres that can be used in the method according to the present invention, it is possible to use microspheres that sediment less, for example non-magnetic latex beads, and that are therefore easier to use, in particular in automated devices.

The term “compound A” is intended to denote in the present description, for all the microsphere populations, any compound present at the surface of said functionalized non-magnetic microspheres as described above, which compound is capable of binding specifically with another compound attached to the surface of the magnetic particles of a ferrofluid, said compound A forming the first member of the specific binding pair, and the other compound forming the second member, said compound A also being characterized in that it cannot bind with the analytes.

The term “ferrofluid” is intended to denote in the present description a stable colloidal suspension of magnetic particles in a liquid carrier. The magnetic particles, the average size of which is approximately 100 Å (10 nm), are coated with a stabilizing dispersing agent (surfactant) that prevents agglomeration of the particles even when a strong magnetic field gradient is applied to the ferrofluid. In the absence of a magnetic field, the magnetic moments of the particles are randomly distributed and the fluid has no clear magnetization.

The magnetic particles of the ferrofluid are surface-coated with said compound forming the second member of the specific binding pair with the compound A at the surface of the microparticles. Thus, these magnetic particles are capable of attaching to the surface of the microspheres by virtue of said binding pair formed, said microspheres in this way being magnetized.

In a particular embodiment of the invention, the method is characterized in that said binding pair formed between the compound A and the second member attached to the surface of the ferrofluids is preferably chosen from the group consisting of the specific binding pairs of type biotin/avidin or biotin/streptavidin, enzyme/cofactor, lectin/carbohydrate and antibody/hapten.

As an example of ferrofluid, mention may in particular be made, but without being limited thereto, of FF-SA (ferrofluids-streptavidin) from Molecular Probes Europe (Leiden, N L) (Ref. C-21476, batch #71A1-1).

The amount of magnetizable material (magnetic particles of the ferrofluid) to be placed on each population of microspheres can therefore be readily controlled by modifying the amount of attachment points (formation of binding pairs) per population of microbeads.

In addition, the sedimentation of the ferrofluids is also very limited, which facilitates the handling and the homogeneity of sampling of the suspensions.

The magnetization of the microparticles is carried out according to a conventional protocol using a magnet (for example, Dynal MPC).

The term “compound B” is intended to denote in the present description, for each of the microsphere populations, any compound present at the surface of the microspheres of one of the populations, which functionalized non-magnetic microspheres are as described above, which compound is capable of forming a specific bond with one of the analytes, the detection and/or quantification of which is being sought, which may be contained in the sample.

As an example of compound B, mention may be made, but without however being limited thereto, of proteins or fragments of structures derived therefrom, receptors, polyclonal or monoclonal antibodies or fragments thereof, monovalent antibodies, single-stranded or double-stranded nucleic acids or any derived fragment or construct, and also combinations of several of these components. As examples of specific binding between the compound B and an analyte, mention may be made of antigen-antibody or ligand-membrane receptor coupling, or hybridization between nucleic acids, the nucleotide sequence of the compound B grafted to the surface of the microspheres then being complementary to that of the analyte.

Preferably, the compound B is a protein or a fragment thereof, capable of recognizing one of said analytes, for instance a polyclonal or monoclonal antibody or a fragment thereof, directed specifically against the analyte intended to be detected and/or quantified, or conversely, the compound B may be an antigen or hapten capable of recognizing an antibody that is intended to be detected and/or quantified, or else a ligand specific for a receptor, an enzyme specific for a cofactor, or a nucleic acid capable of hybridizing specifically with a nucleic acid that is intended to be detected and/or quantified.

When the compound B or C is an antibody, reference may be made, for the preparation of polyclonal or monoclonal antibodies, or fragments thereof, or else recombinant antibodies, to the techniques well known to those skilled in the art, which techniques are in particular described in the “Antibodies” manual (Harlow et al., Antibodies: A Laboratory Manual, Cold Spring Harbor Publications pp. 726, 1988) or to the technique for preparation from hybridomas described by Köhler et al. (Köhler and Milstein, Nature, 256: 495-497, 1975). Specific antibodies can be obtained, for example, from serum or from a cell of an animal immunized specifically against antigens.

The expression “antibody capable of specifically recognizing antigens” is intended to denote in particular the antibody fragments comprising any fragment of said antibody capable of binding specifically to the epitope of said antigen to which the antibody from which the fragment is derived binds. Examples of such fragments include in particular single-chain antibodies (scFv) or monovalent Fab or Fab′ fragments and divalent fragments such as F(ab′)2, which have the same binding specificity as the antibody from which they are derived. These antibody fragments can be obtained from the polyclonal or monoclonal antibodies by methods such as digestion with enzymes, for instance pepsin or papain and/or by cleavage of the disulfide bridges by chemical reduction. These antibodies, or the fragments thereof, may also be obtained in another way, by genetic recombination (recombinant antibodies).

Thus, according to a particular embodiment of the invention, the method is characterized in that said compound B is chosen from the group consisting of proteins and nucleic acids.

According to a particular embodiment, the compound B present at the surface of the microspheres of each of the populations is an antibody and the analytes to be detected and/or quantified are antigens.

Step d) of the method according to the present invention is carried out only when said analytes intended to be identified and/or quantified have not been labeled beforehand, as was described above. In this case, said step d) is necessary for carrying out the subsequent step e) of detection and/or quantification. It involves a solution comprising at least one conjugate capable of binding specifically to one of said analytes intended to be detected and/or quantified in the sample.

Said conjugate of the method according to the present invention comprises a compound C capable of recognizing and of binding specifically with one of said analytes, and a label that is associated therewith.

Preferably, the method according to the invention is characterized in that the compound C of said conjugate used in step d) is a compound chosen from the group consisting of proteins and nucleic acids, when said analytes intended to be detected and/or quantified in the sample are, respectively, proteins or nucleic acids.

When step d) of the method according to the invention is carried out, it is preferably followed by at least one step consisting in washing by magnetic separation, which step is necessary for separating the labeled magnetized microspheres from those carrying only said analytes at their surface.

Step e) of the method according to the present invention can be carried out according to any automated electronic or optical method for detecting and counting particles. Flow cytometry is particularly suitable for this type of analysis.

This method, widely used today, makes it possible to carry out light intensity measurements of very high sensitivity on microspheres in suspension in a reaction medium. These measurements are carried out individually on each microsphere, at high throughput (several hundred to several thousand microspheres analyzed/examined per second), which makes it possible, in a few tens of seconds, to carry out these measurements on a large number of microspheres. Several light parameters can be measured simultaneously on each of the microspheres:
i) laser light scattering/diffraction parameters in order to characterize/evaluate the size and the structure (granularity, density) thereof, firstly, and
ii) secondly, several fluorescence parameters, that can be differentiated by their wavelengths and are generally associated with the presence of fluorochromes, or fluorescent labels, intrinsically present in the microsphere, or associated with the specific binding of conjugates.

In practice, flow cytometry (FCM) consists in passing the microspheres, in suspension in a liquid, one by one in front of a focused laser beam, and measuring, on each microsphere individually, firstly the laser light scattering/diffraction and, secondly, the associated fluorescence signals. All this information is provided to the user in the form of frequency distributions (histograms) in which said user easily locates subpopulations that are homogeneous with respect to one or more of the parameters under consideration (for example, the size or a fluorescence). One (or more) fluorescence parameter(s) can be used, in addition to the size/structure parameters, to group together individuals belonging to the same type of microspheres (multiplex assay). One (or more) other fluorescence parameter(s) can be used as a visualizing agent, the intensity of which is directly proportional to the amount of analytes present on the type of microsphere under consideration.

An example of flow cytometry that can be used in step e) is the FACS (fluorescence-activated cell sorter) technique, which consists of an electron system for separating microspheres according to their size and the intensity of the fluorescence that they emit after various labelings. The device prepares microdrops of the microsphere suspension, which are diluted so as to contain only one microsphere. The microdrop passes in front of a laser ray light beam and the microspheres are analyzed (histogram) and separated on the basis of their fluorescence and/or of their size.

Among the labels that can be used for labeling the compound C or for labeling said analytes intended to be detected and/or quantified in the sample, preference is given to fluorescent labels such as, in particular, but without being limited thereto, fluorescein and its derivatives, such as fluorescein isothiocyanate (FITC), or else allophycocyanin (APC), phycoerythrin-cyanin 5 (PC5) and phycoerythrin (PE), R-phycoerythrin (R-PE), or alternatively rhodamine and its derivatives, coumarin and its derivatives, luciferase and its derivatives, chromomycin, mithramycin, GFP (for “green fluorescent protein”), eGFP (for “enhanced green fluorescent protein”), RFP (for “red fluorescent protein”), BFP (for “blue fluorescent protein”), eBFP (for “enhanced blue fluorescent protein”), YFP (for “yellow fluorescent protein”), eYFP (for “enhanced yellow fluorescent protein”), dansyl, umbelliferone, ethidium bromide, acridine orange, thiazole orange, propidium iodide (PI), etc.

Thus, preferably, the method according to the invention is characterized in that the label(s) used is (are) fluorescent.

More preferably, the method according to the invention is characterized in that the detection and/or the quantification of said label in step e) of the method is carried out by flow cytometry.

The techniques for coupling these labels are well known to those skilled in the art and will not be developed in the present description. However, for certain analytes, conjugates comprising such fluorescent labels directed against the analyte intended to be detected and/or quantified can be found commercially.

In such a method for the detection and/or multiplex quantification of several analytes, a range of labels that can be specifically detected and/or quantified simultaneously are preferably used, preferably fluorescent labels. Particularly preferably, flow cytometry will be used for the direct and simultaneous detection and/or quantification of said range of fluorescent labels.

Thus, the suspension of microsphere populations may comprise a first population n₁ in which the microspheres of which it is composed will each have the compound B₁ attached to their surface, a second population n₂ in which the microspheres of which it is composed will each have the compound B₂ attached to their surface, a third population n₃ in which the microspheres of which it is composed will each have the compound B₃ attached to their surface, and so on. Thus, the analyte₁ will be recognized by the compound B₁, the analyte₂ will be recognized by the compound B₂, the analyte₃ will be recognized by the compound B₃, and so on.

According to a particular embodiment, the method according to the invention is characterized in that at least two of said populations also have at least one intrinsic physical characteristic that makes it possible to differentiate them from one another.

Those skilled in the art may have available microsphere populations exhibiting intrinsic physical characteristics that make it possible to differentiate them from one another, thus making it possible to increase the number of different analytes to be detected and/or quantified in a sample, it being possible for said intrinsic physical characteristics to be differentiatable by means of their size and/or their optical properties (fluorescence specific for each population).

Thus, preferably, the intrinsic physical characteristic of the microspheres of the method according to the present invention that makes it possible to differentiate the at least two microsphere populations is the size and/or an optical property of said microspheres.

More preferably, the method according to the invention is characterized in that the microspheres have a size of between 0.3 and 100 μm.

Even more preferably, the method according to the invention is characterized in that the microspheres have a size of between 1 and 20 μm.

These size ranges correspond as much as possible to the analytical size range of common flow cytometers. Within these diameter ranges, and on the condition of not imposing additional constraints that are too rigid (magnetism, fluorescence), it is easy to find beads that can be distinguished from one another via only their size parameter, measured by the parameter called forward light scatter (FS or FLS), so as to form several distinct groups (for example, 1, 3, 5, 8, 10 and 15 μm). Some of these diameters are also available in a fluorescent version, or even with several differentiatable intensity levels.

According to another embodiment, the method according to the invention is characterized in that the optical property is the emission wavelength and/or the fluorescence intensity of said microspheres.

Thus, it is possible to differentiate the n populations of microspheres of said suspension from one another.

Thus for example, the ranges QuantumPlex™ provided by Bang's Labs (Fishers, US) and CytoPlex™ provided by Duke Scientific (Palo Alto, US) make it possible to obtain, for a given size, subfamilies of beads that can be differentiated through their level of intensity in red fluorescence, up to 10 levels for beads of 4 μm (CytoPlex) or 2×5 levels for beads of 4.4 and 5.5 μm (QuantumPlex). Assuming the beads to be available, ad hoc, in the abovementioned diameters, and each with 5 levels of intensity of red fluorescence, the multiplex detection possible according to the invention comes to 6×5=30 analytes. Advantageously, the method according to the invention allows the rapid detection and/or quantification of at least 6 different analytes.

The specificity required for the populations of conjugates used depends on the complexity of detection and/or quantification step e), and on the type and number of analytes sought. It is thus possible to use just a single conjugate population comprising a single type of label, said conjugate then having a ubiquitous visualizing function. In this case, the specificity is provided only by the selectivity of each trapping bead. Conversely, it is also possible to use as many different conjugate populations as there are analytes to be assayed. Depending on the test to be carried out, those skilled in the art may of course choose the suitable alternatives between these two extreme variants.

According to an advantageous embodiment, the method according to the invention is characterized in that said analytes are nucleic acids and in that, in step a), the compound B is a nucleic acid capable of hybridizing specifically with one of said analytes.

Preferably, said analytes are PCR products.

More preferably, said PCR products are obtained labeled.

Such PCR products, where appropriate labeled, have been previously described. The functionalized non-magnetic microspheres used in this embodiment carry a compound B of oligonucleotide type at their surface, which compound B is complementary to one of the amplification products sought. When the products are labeled beforehand, step d) consisting in bringing the magnetized microspheres into contact with conjugates is not necessary. The labeling at the surface of the microspheres is provided by the label carried by the PCR products that the microspheres have trapped.

Even more preferably, the method according to the invention is characterized in that the compound C of said conjugate used in step d) is a nucleic acid capable of hybridizing specifically with one of said analytes.

Such a type of conjugate may be used, for example, for detecting and/or quantifying several analytes, such as, in particular, genomic material not amplified beforehand, or derivatives not amplified beforehand, for instance fragments generated by enzymatic cleavage using restriction enzymes for this genomic material.

The signal amplification step can then be provided by the step consisting of ligation (or linking) between the compound B of the microspheres and the compound C of the conjugates.

Thus, a subject of the invention is also the use of the method according to the invention, for the detection and/or multiplex quantification of SNPs (Single Nucleotide Polymorphisms).

In this case, the method of the invention is preferably used in combination with the OLA technique, for “Oligonucleotide Ligation Assay”, based on the action of a ligase that links two adjacent oligonucleotides covalently only on the condition that they are completely complementary to the template DNA strand (Landegren et al., Proc. Natl. Acad. Sci. US 1990;87:8923-8927; U.S. Pat. No. 4,988,617). As indicated above, the template DNA strand can be either an amplicon (fragment amplified by PCR/Polymerase Chain Reaction or another enzymatic amplification reaction), or genomic material not amplified beforehand or its derivatives not amplified beforehand, for example fragments generated by enzymatic cleavage with restriction enzymes for this genomic material.

Thus, preferably, the use according to the invention is characterized in that the detection and/or multiplex quantification of SNPs is carried out by the OLA method.

In this variant, the conjugate within the meaning of the general definition above is a visualizing probe (compound C) carrying a fluorescent label. The ligation step constitutes an additional step between step d) and step e) of said method.

The protocol described hereinafter and shown diagrammatically in FIG. 1 illustrates the general principle of the invention. This embodiment relates to the detection of three different antigens. Of course, on this basis, many variants may be envisioned by those skilled in the art, depending on the nature and the number of different analytes to be detected and/or quantified, the sensitivity of the test, the rate at which the test is carried out, the material used, etc. These variants may, for example, concern the number and/or the size and/or the optical properties of the functionalized microspheres, the number of ligands specific for the analyte sought, attached to their surface (compound B), the magnetization step which can be repeated several times alternating with successive washes, the nature of the label bound to the conjugate, which can be the same for all the conjugates of the mixture or can be very different according to the specificity of the conjugates, etc.

Populations of functionalized microspheres are brought into contact with the sample that it is desired to test. In the interests of simplicity and clarity, it is considered, in this description, that there are three populations of latex microspheres (three analytes to be detected and/or quantified) of different sizes. Each of the populations carries at its surface a compound B which is a trapping antibody specific for one of the three analytes sought. In this illustration, it is also considered that the compounds A are biotin molecules which are grafted to the surface of the microspheres so as to allow the binding of the magnetic particles of the ferrofluid via biotin-streptavidin binding. When the microspheres are mixed with the sample, each of the analytes sought will bind to the population carrying the specific antibody. The magnetic particles of the ferrofluid coupled to streptavidin are then added to the medium and will bind to the surface of the microspheres. The microspheres thus made magnetizable can be separated from the other interfering products of the medium by means of one or more magnetization steps alternating with one or more steps consisting in washing with an appropriate buffer.

The microsphere populations are subsequently brought into contact with a mixture of three types of conjugates, each type of conjugate being represented in this scheme by a compound C which is a specific antibody carrying a fluorescent label.

After a phase consisting of incubation of the microspheres with the conjugate solution, sufficient to allow the binding of said conjugates to their specific analyte, itself retained at the surface of the microspheres, the microspheres associated with the fluorochrome can be analyzed. In the present case, they are analyzed by flow cytometry according to their size.

The conjugates are generally used in excess so as to ensure optimal labeling of the analytes bound to the microspheres. As a result, before carrying out the analysis of the microspheres, it is preferable to isolate the latter from the medium containing excess conjugates that have remained in suspension. This separation is advantageously carried out once again with one or more series(s) of magnetization and washing.

As has already been indicated, an advantageous variant of the method according to the invention is directed toward the detection and/or quantification of n nucleic acids. In its simplest embodiment, this variant takes place according to the protocol hereinafter, shown diagrammatically in FIG. 2.

In this case, the analysis relates to three fluorescent PCR products using beads of different sizes. Each of the populations of functionalized microspheres is coated with a compound B which is an oligonucleotide complementary to one of the PCR products (trapping probe) and no longer with a specific antibody. The steps consisting in mixing and magnetizing the microspheres are identical to those described in the preceding embodiment. After the magnetization and washing steps, the microspheres that trapped fluorescent products at their surface are analyzed by flow cytometry.

A subject of the present invention is also a kit for the detection and/or multiplex quantification of analytes that may be contained in a sample, characterized in that it comprises a suspension of populations of functionalized non-magnetic microspheres, said microspheres carrying at their surface:
a) a reagent 1 comprising:
   a compound A forming a first member of a binding pair;
   a compound B capable of forming a specific binding with one of said analytes of the sample, and
b) a reagent 2 comprising a ferrofluid which contains magnetic particles carrying at their surface a second binding member capable of forming a specific binding pair with said compound A; and
c) a reagent 3 comprising a solution of at least one conjugate, said conjugate comprising a compound C capable of reacting specifically with said analytes, and a label capable of being detected.

More preferably, the kit according to the invention also comprises:
a reagent 4 comprising said analytes that may be contained in a sample.

Even more preferably, the kit according to the invention also comprises:
a reagent 5 composed of a dilution buffer; and
a reagent 6 composed of a washing buffer.

Finally, according to another even more preferred embodiment, the kit according to the invention also comprises:
a reagent 7 comprising a buffer for neutralizing the aggregation of the various microspheres.

Said buffers are, for example, PBS-based buffers, for instance PBS/Tween 20.

The reagent 7 will more particularly be used when the compound A at the surface of the microspheres is a biotin-type compound. Said neutralization buffer makes it possible to prevent aggregation of the various microspheres coated with compound A during the magnetization. In this case where biotinylated microspheres are involved, the neutralization buffer consists, for example, of an aqueous solution of biotin.

As has already been indicated above, and as will emerge from reading the examples hereinafter, the method of the invention makes it possible to simultaneously identify several agents in a sample, in relatively quick times.

The figure legends and examples that follow are intended to illustrate the invention without in any way limiting the scope thereof.

### FIGURE LEGENDS

FIG. 1: Scheme of the principle of the method of detection and/or quantification (multiplex assay) according to the invention applied to the detection and/or quantification of three antigens using beads of different sizes.

FIG. 2: Scheme of the principle of the method according to the invention applied to the multiplex assaying of three fluorescent PCR products using beads of different sizes.

FIG. 3: Scheme of the principle of the multiplex assaying method according to the invention applied to molecular genetics for searching for SNPs.

FIG. 4: Scheme illustrating the same principle as above, in which an additional degree of specificity is obtained in terms of the grafting of the visualizing probe.

FIGS. 5A, 5B, 5C: FCM analysis of a mixture of beads of 3, 8, 10 and 15 μm according to size.

R1: Estapor 3 μm+Polymer Laboratories 8 and 15 μm+Dynal Particles 10 μm;

R2 and R6: Estapor 3 μm; R3 and R7: Polymer Laboratories 8 μm; R4 and R8: Dynal Particles 10 μm; R5 and R9: Polymer Laboratories 15 μm.

FIGS. 6A, 6B, 6C and 6D: FCM analysis of a mixture of beads of 3, 4.4, 8, 10 and 15 μm according to size and of fluorescence.

R1: Estapor 3 μm+Bangs QuantumPlex 4.4 μm+Polymer Laboratories 8 and 15 μm+Dynal Particles 10 μm; R2 and R6: Estapor 3 μm+Bangs QuantumPlex 4.4 μm; R3 and R7: Polymer Laboratories 8 μm;

R4 and R8: Dynal Particles 10 μm; R5 and R9: Polymer Laboratories 15 μm; R10: Estapor 3 μm; R11: Bangs QuantumPlex #3; R12: Bangs QuantumPlex #5 (R10, R11 and R12 are contained within R2).

FIGS. 7A and 7B: FCM analysis of a mixture of beads of 4.4, 8, 10 and 15 μm that are initially non-magnetic.

FIG. 8: Change in distribution, by FS LOG, of the various bead-biotin populations during binding of the FF-SAs.

FIG. 9: Assaying of B. globigii on anti-B. globigii 15 μm microsphere.

FIGS. 10A and 10B: Assaying of ovalbumin on anti-ovalbumin 4.4 μm QuantumPlex #3 microsphere.

FIG. 11: FCM analysis on duplex mixture:

The 2 types of beads are distinguished through their size by double scatter analysis, μS-FV (diameter 6.7 μm), gated on region R1), and μS-FII (diameter 9.6 μm), gated on region R2. This selective analysis based on size is repeated in all the figures that follow.

FIGS. 12 and 13: FCM analysis on a duplex mixture of a doubly positive test:

The beads are brought into contact with the two O.N. representing the 2 genes simultaneously.

FIG. 12 shows the levels of fluorescence as a function of size, μS-FV (R4 region) and μS-FII (R3 region).

FIG. 13 shows, in superposition, the respective green fluorescence histograms of each type of beads, solid curve for the μS-FV bead and empty curve for the μS-FII bead. The mean green fluorescence intensities (MFI) are measured in each of the windows M1 and M2.

FIGS. 14 and 15: FCM analysis on a duplex mixture; negative control:

The beads brought into contact with the two O.N. simultaneously are dehybridized by heat, showing the nonspecific background noise labeling.

FIG. 14 shows the fluorescence levels as a function of size, μS-FV (R4 region) and μS-FII (R3 region).

FIG. 15 shows, in superposition, the respective green fluorescence histograms of each type of beads, solid curve for the μS-FV bead and empty curve for the μS-FII bead. The MFI are measured in each of the windows M1 and M2.

FIGS. 16 and 17: FCM analysis on a duplex mixture of a test single-positive for FV:

The beads are brought into contact with a single O.N., corresponding to FV.

FIG. 16 shows the fluorescence levels as a function of size, μS-FV (R4 region) and μS-FII (R3 region).

FIG. 17 shows, in superposition, the respective green fluorescence histograms of each type of beads, solid curve for the μS-FV bead and empty curve for the μS-FII bead. The MFI are measured in each of the windows M1 and M2.

FIGS. 18 and 19: FCM analysis on a duplex mixture of a test single-positive for FII:

The beads are brought into contact with a single O.N., corresponding to FII.

FIG. 18 shows the fluorescence levels as a function of size, μS-FV (R4 region) and μS-FII (R3 region).

FIG. 19 shows, in superposition, the respective green fluorescence histograms of each type of beads, solid curve for the μS-FV bead and empty curve for the μS-FII bead. The MFI are measured in each of the windows M1 and M2.

FIG. 20: The critical base (SNP specificity) is carried by each of the allele-specific visualizing probes that each carry a different fluorochrome (or a hapten/tag that can be visualized with a fluorescent anti-tag MAb). This system takes advantage of multi-color analyses that can be carried out by FCM. Each type of bead allows the differential detection of a mutation.

FIG. 21: The critical base (SNP specificity) is carried by each of the allele-specific trapping probes each coupled to a different type of bead (differentiated, for example, by the size). For signal analysis by FCM, this system calls for only one fluorescence, making it possible either to use a simpler and less expensive device, or to take advantage of other colors for differentiating the families of beads from one another, in multi-color analyses.

FIG. 22: FCM analysis on a duplex mixture:

The 2 types of beads are distinguished through their size by double scatter analysis, μS-FVwt (diameter 6.7 μm), gated on the R1 region) and μS-FVmut (diameter 9.6 μm, gated on the R2 region). This selective analysis based on size (FS) and granulosity (SS) is repeated in all the figures that follow.

FIGS. 23 and 24: FCM analysis on a duplex mixture of a negative control:

The beads are brought into contact with amplicons that are not specific for the mutation studied, but serve as a negative control in the presence of the “Ampli-Mix” PCR mix.

FIG. 23 shows the fluorescence levels as a function of size, μS-FVwt (R4 region) and μS-FVmut (R3 region).

FIG. 24 shows, in superposition, the respective green fluorescence histograms of each type of beads, solid curve for the μS-FVwt bead and empty curve for the μS-FVmut bead. The mean fluorescence intensities (MFI), measured in the windows M1 and M2, are indicated for each type of beads.

FIGS. 25 to 28: FCM analysis on a duplex mixture of a single-positive test on a wild-type allele:

The beads are brought into contact with amplicons of a single allele (FVwt).

FIG. 25 shows the fluorescence levels as a function of size, μS-FVwt (R4 region) and μS-FVmut (R3 region).

FIG. 26 shows, in superposition, the respective green fluorescence histograms of each type of beads, solid curve for the μS-FVwt bead and empty curve for the μS-FVmut bead.

FIG. 27 shows, on μS-FVwt, the histograms of the test (right-hand curve) and of the BN (left-hand curve, repeat of FIG. 24).

FIG. 28 shows, on μS-FVmut, the histograms of the test (right-hand curve) and of the BN (left-hand curve, repeat of FIG. 24).

FIGS. 29 to 32: FCM analysis on a duplex mixture of a single-positive test on a mutant allele:

The beads are brought into contact with amplicons of a single allele, in this case FVmut.

FIG. 29 shows the fluorescence levels as a function of size, μS-FVwt (R4 region) and μS-FVmut (R3 region).

FIG. 30 shows, in superposition, the respective green fluorescence histograms of each type of beads, solid curve for the μS-FVwt bead and empty curve for the μS-FVmut bead.

FIG. 31 shows, on μS-FVwt, the histograms of the test (right-hand curve) and of the BN (left-hand curve, repeat of FIG. 24).

FIG. 32 shows, on μS-FVmut, the histograms of the test (right-hand curve) and of the BN (left-hand curve, repeat of FIG. 24).

FIGS. 33 to 36: FCM analysis on a duplex mixture of a double-positive test:

The beads are brought into contact with amplicons of the two alleles simultaneously.

FIG. 33 shows the fluorescence levels as a function of size, μS-FVwt (R4 region) and μS-FVmut (R3 region).

FIG. 34 shows, in superposition, the respective green fluorescence histograms of each type of beads, solid curve for the μS-FVwt bead and empty curve for the μS-FVmut bead.

FIG. 35 shows, on μS-FVwt, the histograms of the test (right-hand curve) and of the BN (left-hand curve, repeat of FIG. 24).

FIG. 36 shows, on μS-FVmut, the histograms of the test (right-hand curve) and of the BN (left-hand curve, repeat of FIG. 24).

FIG. 37: In FIG. 37, as in FIG. 21, the critical base (SNP specificity) is carried by each of the allele-specific trapping probes, each coupled to a different type of bead (differentiated by size). For the signal analysis by FCM, this system calls for only an analysis by counting of the beads on the basis of the size and structure parameters, making it possible either to use a device that has no fluorescence detector and is therefore less expensive, or to take advantage of other sizes for differentiating the families of beads from one another.

FIG. 38: FCM analysis on a duplex mixture:

The 2 types of beads are distinguished through their size in double scatter analysis, μS-FVwt (diameter 6.7 μm, gated on the R1 region) and μS-FVmut (diameter 9.6 μm, gated on the R2 region). This selective analysis based on size (FS) and granulosity (SS) is repeated in all the figures that follow.

FIGS. 39 and 40: FCM analysis on a duplex mixture of a negative control:

The beads are brought into contact with amplicons that are not specific for the mutation studied, that serve as a negative control in the presence of the “Ampli-Mix” PCR mix.

FIG. 39 shows the levels of the number of μS as a function of size, μS-FVwt (R1 region) and μS-FVmut (R2 region).

FIG. 40 shows, in superposition, the respective histograms of the number of μS of each type of beads. The numbers of μS, measured in the windows M1 and M2, are indicated for each type of beads.

FIGS. 41 and 42: FCM analysis on a duplex mixture of a single-positive test on a wild-type allele:

The beads are brought into contact with amplicons of a single allele (FVwt).

FIG. 41 shows the levels of the number of μS as a function of size, μS-FVwt (R1 region) and μS-FVmut (R2 region).

FIG. 42 shows, in superposition, the respective histograms of the number of μS of each type of beads. The numbers of μS, measured in the windows M1 and M2, are indicated for each type of beads.

FIGS. 43 and 44: FCM analysis on a duplex mixture of a single-positive test on a mutant allele:

The beads are brought into contact with amplicons of a single allele, in this case FVmut.

FIG. 43 shows the levels of the number of μS as a function of size, μS-FVwt (R1 region) and μS-FVmut (R2 region).

FIG. 44 shows, in superposition, the respective histograms of the number of μS of each type of beads. The numbers of μS, measured in the windows M1 and M2, are indicated for each type of beads.

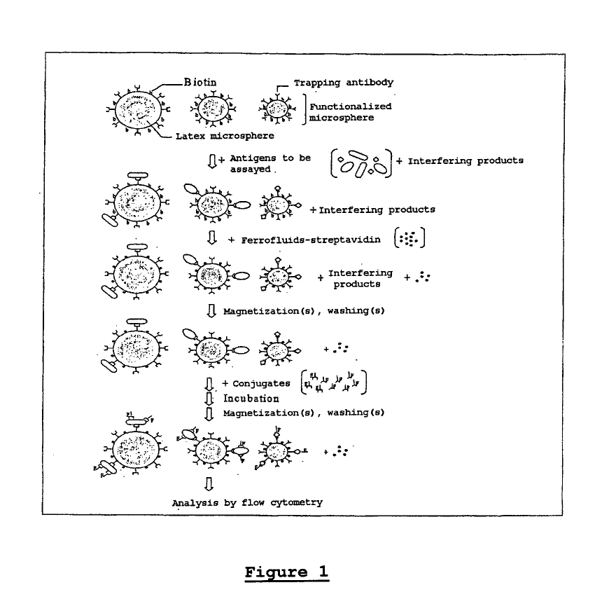

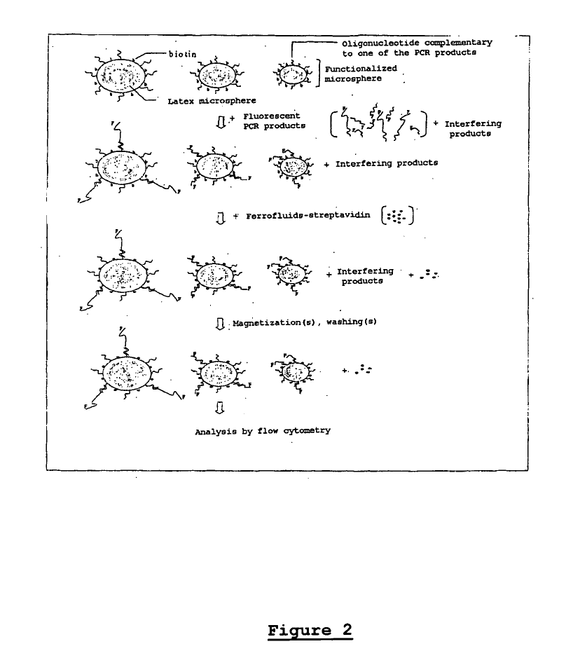

### EXAMPLES

### Example 1

### Recognition of Families of Microspheres (μS) as a Function of Size by FCM

The microspheres listed below were mixed in similar proportions and the mixture was analyzed on an EPICS XL flow cytometer (Coulter) (FIG. 5).

The flow cytometry analysis of a mixture of 6 populations of beads of sizes 2, 3.1, 6, 7.6, 10.2 and 15.1 μm showed that the singlets of the various populations of beads could be differentiated by double scatter analysis. The parameters are measured after logarithmic amplification (FS log/SS log) (FS for Forward light Scatter; SS for Side light Scatter).

List of the 6 populations of microspheres used:

| | | Diameter | |
|---|---|---|---|
| Reference | Supplier | (μm) | Polymer |
| Dynospheres Calibration | Dyno Particles | 15.1 | Polystyrene |
| Kit | (Dynal Biotech) | | (PS) |
| Dynospheres Calibration | Dyno Particles | 10.2 | PS |
| Kit | (Dynal Biotech) | | |
| Uniform Latex Particles | Seradyn | 7.6 | PS 98%/ |
| | | | Divinylbenzene |
| | | | (DVB) 2% |
| Sphero Polystyrene | Spherotech, Inc. | 6 | PS |
| Particles | | | |
| Microspheres Estapor | Estapor | 3.1 | PS |
| White 3 μm | (Merck Eurolab) | | |
| Dynospheres Calibration | Dyno Particles | 2 | PS |
| Kit | (Dynal Biotech) | | |

The flow cytometry analysis of a mixture of 4 populations of beads of approximate sizes 3, 8, 10 and 15 μm showed that the singlets of the various populations of beads could be readily differentiated by FS log/SS log and that the possible multiplets did not represent a hindrance.

List of the 4 populations of microspheres used:

| | | | Diameter | CV | |
|---|---|---|---|---|---|
| Reference | Batch No. | Supplier | (μm) | (%) | Polymer |
| PL-Microspheres | SP-1444 | Polymer | 14.57 | 2.92 | Polystyrene (PS) |
| Plain White 15 μm | | Laboratories | | | |
| Dynospheres Q-561 | Q-561 | Dyno Particles | 10.1 | 1.00 | PS 94.5%/ |
| | | (Dynal Biotech) | | | DVB 5.5% |
| PL-Microspheres | SP-1436 | Polymer | 7.97 | 2.29 | PS |
| Plain White 8 μm | | Laboratories | | | |
| Microspheres | 285 | Estapor | 3.1 | ND | PS |
| estapor White 3 μm | | (Merck Eurolab) | | | |
| R 94-52 | | | | | |

See FIGS. 5A to SC

### Example 2

### Differentiation of Multiple (6) Families of Microspheres by FCM as a Combined Function of Size and of a Fluorescence

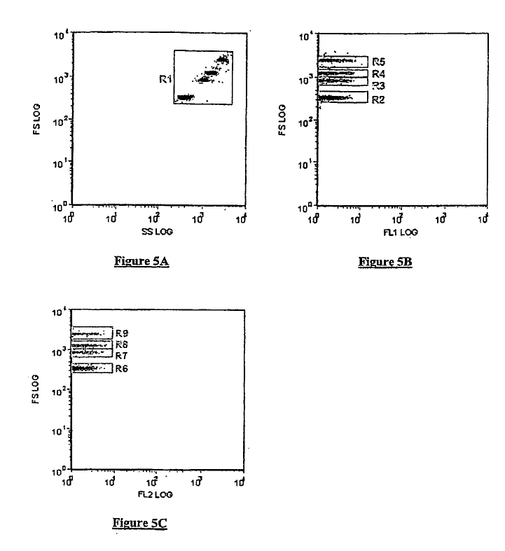

Microspheres of 4.4 μm carrying a red fluorescence (measured on the FL4 detector) were added to the mixture of example 1. The combination, with the mixture described above, of microspheres of 4.4 μm makes it possible to recognize 2 additional families; the difference in forward scatter (log FS) between the microspheres of 3 μm and those of 4.4 μm remains too small to be readily discriminated (cf. FIG. 5A and B). The introduction of FL4 as an associated parameter allows complete discrimination of the microspheres of 4.4 μm with respect to all the others (3 μm in particular), and for the 2 groups of 4.4 μm microspheres with respect to one another (FIG. 6).

### Example 3

### Functionalization of 8, 10 and 15 μm Microspheres by Passive Adsorption

IgG purification:

Polyclonal sera from rabbits immunized against the model bacteria B. globigii, B. pseudomallei or Y. pestis or against the model soluble antigens ovalbumin (OVA) and ricin A chain (Ricin) were generated. The rabbit immunoglobulins G (IgGs) were purified by affinity chromatography on protein G.

Briefly, 50 ml of diluted serum were loaded onto a column containing 5 ml of protein G Sepharose 4 fast flow (Pharmacia) pre-equilibrated in Na₂HPO₄ buffer, pH=7. The attached IgGs were subsequently eluted in 0.1 M glycine/HCl buffer, pH 2.7, and then immediately neutralized with Tris/HCl buffer, pH=9.

The eluates were dialyzed against 150 mM PBS buffer, pH=7.2, at 4° C., and were then concentrated by reverse osmosis. The IgG concentrations were estimated by reading the absorbance at 280 nm (ε_{0.1%} at 280 nm=1.41).

Preparation of 8, 10 and 15 μm beads:

1 ml of latex beads containing 10% solid material (i.e. 100 mg of latex), 8 μm (Polymer Laboratories), 10 μm (Dynal Particles) or 15 μm (Polymer Laboratories) in diameter, were centrifuged for 10 min at 500 g. After the removal of 600 μl of supernatant, 2 ml of PBS buffer/0.25% Triton X-100/0.09% NaN₃ (PBS/Triton) were added. The beads were incubated at ambient temperature for 10 min, washed with 2.4 ml of PBS buffer, then resuspended in a final volume of 6 ml of this same buffer and placed at 4° C. for 30 min.

Antibody (Ab) preparation:

The Ab were diluted in PBS buffer to a concentration of 200 μg/ml in a volume of 1 ml and placed at 4° C. for 30 min. Two tubes containing 1 ml of 150 mM PBS/0.1% BSA/0.09% NaN₃ (PBS/BSA) were also provided for in order to obtain nonloaded beads.

The BSA-biotin (Sigma, Ref. A-8549) was diluted in PBS buffer to 500 μg/ml in a volume of 1 ml and placed at 4° C. for 30 min.

Bead loading: 1 ml of beads (8, 10 or 15 μm) was added to the various antibody solutions maintained with strong agitation (vortex). The various mixtures were then placed at 4° C. for 12 hours with rotary shaking. After this incubation, the mixtures were centrifuged for 10 min at 500 g. After removal of the supernatant, the loaded beads were biotinylated by incubation at 4° C. for 3 hours in 2 ml of BSA-biotin at 500 μg/ml. After removal of the supernatant, the loaded beads were saturated by incubation for 2 hours in 2 ml of PBS buffer/2% BSA. Two washes in PBS buffer were carried out before taking up the beads with 1 ml of PBS/BA. The suspensions obtained were numbered and the concentrations thereof were adjusted to 2.5×10⁴/μl.

### Example 4

### Functionalization of 4.4 μm Microspheres by Covalent Coupling

The anti-OVA and anti-Ricin Ab were covalently coupled after COOH-activation of the beads (protocol adapted from the Bang's Labs procedure, Ref. TechNote #205: “Covalent Coupling”). The carbodiimide used for activating the beads is 1-ethyl-3-(3-dimethylaminopropyl)carbodiimide HCl (EDC, Pierce—Brebieres, FR—, Ref. 1853160).

Ab preparation:

The anti-OVA (respectively, anti-Ricin) IgGs were diluted in PBS buffer to a concentration of 200 μg/ml in a volume of 1 ml.

Bead activation:

1 ml of Bang's Labs QuantumPlex #5 and #3 latex beads, diameter 4.4 μm, were centrifuged for 10 min at 1900 g. After removal of the supernatant, 2 ml of 0.1 M MES buffer, pH=5.5, were added. After this operation had been repeated, 500 μl of 0.1 M MES buffer/10 mg/ml EDC, pH=5.5, were added. The mixtures thus obtained were placed on a rotary shaker for 15 min, washed twice with 2 ml of PBS buffer, and then taken up in a 1 ml volume of this same buffer.

Coupling of the Ab to the beads: 1 ml of QuantumPlex #5 beads was added to the anti-Ricin IgG solution maintained under strong agitation (vortex). The same operation was carried out by mixing the QuantumPlex #3 beads with the anti-OVA IgG solution previously prepared. The various mixtures were then placed at ambient temperature for 4 h with rotary shaking.

Biotinylation of the beads:

The mixtures were centrifuged for 10 min at 1900 g. After removal of the supernatant, the loaded beads were biotinylated by incubation overnight at 4° C. in 2 ml of PBS/0.05% BSA-biotin/30 mM Glycine.

The biotin-loading of the beads was subsequently verified by FCM after labeling with Streptavidin-PE (Sigma, Ref. S-3402).

Saturation of the beads:

Subsequent to this incubation, the beads were centrifuged for 10 min at 1900 g. After removal of the supernatant, the loaded beads were saturated by incubation for 30 min in 2 ml of PBS buffer/2% BSA. Washing in PBS buffer was carried out before taking up the beads with 1 ml of PBS/BA. The suspensions obtained were numbered and the concentrations thereof were adjusted to 2.5×10⁴ beads/μl.

### Example 5

### Magnetic Isolation and Differential Recognition of 4.4 μm, 8, 10 and 15 μm microspheres initially non-magnetic.

1. Aim

To demonstrate the possibility of isolating, by magnetization, latex beads that are initially non-magnetic but that become magnetic when there is binding of the magnetic particles of a ferrofluid via streptavidin/biotin binding.

To show that this binding does not result in any overlapping of the various categories of beads in FS LOG/SS LOG.

To show that the bead recovery yields are sufficiently high to allow cytometric analysis.

2. Materials

Mixture of functionalized beads (5000 beads/μl of each specificity) composed of:

4.4 μm QuantumPlex #3 beads/anti-OVA Ab/BSA-biotin (batch # 682).
4.4 μm QuantumPlex #5 beads/anti-Ricin Ab/BSA-biotin (batch # 681).
8 μm Polymer Laboratories beads/anti-Y. pestis Ab/BSA-biotin (batch # 041).
10 μm Dyno beads/anti-B. pseudomallei Ab/BSA-biotin (batch # 042).
15 μm Polymer Laboratories beads/anti-B. globigii Ab/BSA-biotin (batch # 043).
   Ferrofluids-streptavidin (FF-SA) Molecular Probes (Ref. C-21476) at 0.5 mg Fe/ml (batch #71A1-1).
   d-Biotin at 200 μg/ml in distilled water.
   PBS buffer/0.1% Tween.
   PBS buffer/BA.
   1 ml IMS tube.
   Dynal magnet.
   5.1 μm Duke XPR green beads (batch #1938).

3. Protocol

790 μl of PBS/0.1% Tween (IMS tube) or 490 μl of PBS/0.1% BSA/0.09% NaN₃ (PBS/BA) (reference tube) are introduced into a 1 ml IMS tube.

10 μl of functionalized bead mixture (i.e. 50 000 beads of each category) are added.

The mixture is vortexed.

| | |
|---|---|
| For the | 30 μl of FF-SA are added. |
| IMS tube | The mixture is vortexed. |
| only | The mixture is placed on a rotary shaker for |
| | 5′. |
| | 100 μl of biotin at 200 μg/ml are added. |
| | The mixture is incubated for 1 min. |
| | The mixture is vortexed. |
| | The tube is magnetized for 2 min 30 sec. |
| | The buffer is removed. |
| | 800 μl of PBS/BA are added. |
| | The tube is magnetized for 2 min 30 sec. |
| | The buffer is removed. |
| | 500 μl of PBS/BA are added. |

15 μl of Duke XPR beads diluted to 1/50 in PBS/0.1% Tween (reference beads for standardizing the number of events counted during the cytometric analysis) are added.

The two tubes are analyzed on a Coulter EPICS XL cytometer as indicated below:

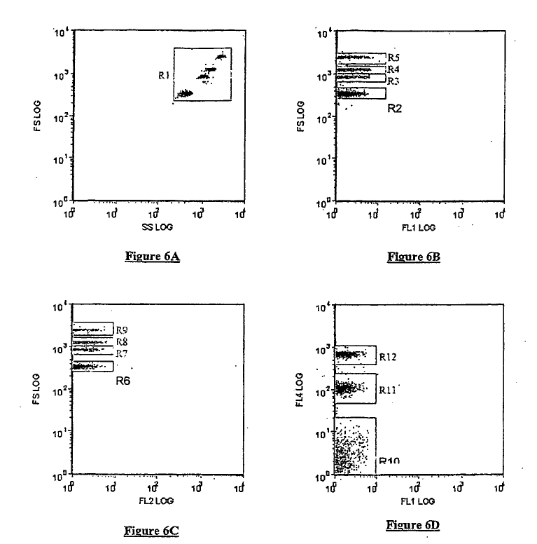

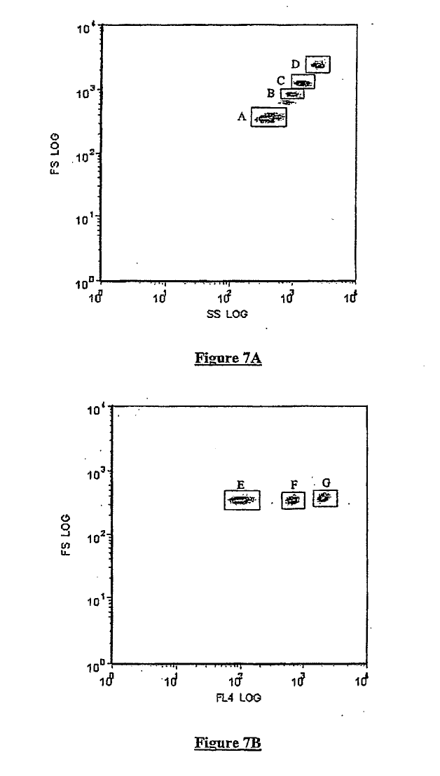

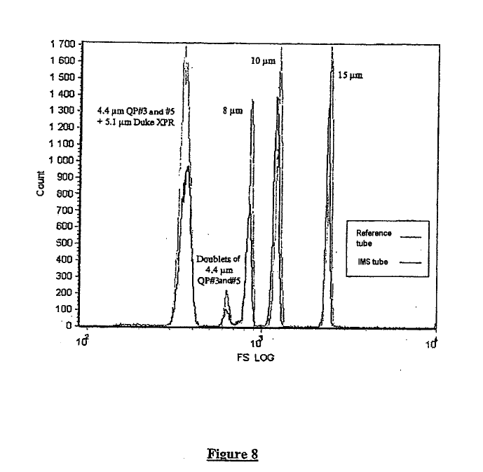

An FS LOG/SS LOG histogram is created. Four analytical regions (A, B, C and D) are created on this histogram (FIG. 7A).

Regions B, C and D are placed on the 8, 10 and 15 μm beads, respectively.

Region A is placed on the population composed of the counting beads (5.1 μm, Duke XPR) and of the 4.4 μn trapping beads.

An FS LOG/FL4 LOG histogram gated on window A (FIG. 7b) is created.

Three analytical regions (E, F and G) are created.

Regions E and F are placed on the populations of 4.4 μm QuantumPlex #3 and #5 beads, respectively. Region G is placed on the population of counting beads (Duke XPR).

A monoparametric histogram gated on region G is created. An automatic analysis stop at 10 000 events is placed on this histogram.

The number of events counted in regions B, C, D, E and F is recorded. The recovery yields for each category of beads are calculated by dividing the number of events counted on the IMS tube by that counted on the reference tube.

4. Results

4.1. Change in distribution, by FS LOG, of the various biotin-bead populations during binding of the FF-SA (FIG. 8).

The binding of the FF-SA to the biotin-BSA/beads results in a slight decrease in the FS. This change does not result in any overlapping of the various bead populations.

4.2. Recovery yields

The recovery yields (% of beads recovered) obtained in 3 different assays, under the conditions disclosed in paragraph 3, are disclosed in the table below.

| | Category of beads/BSA-biotin | | | | |
|---|---|---|---|---|---|
| | 4.4 μm QP#3 | 4.4 μm QP#5 | 8 μm | 10 μm | 15 μm |
| Mean | 52.3 | 50.5 | 69.3 | 93.3 | 90.8 |
| Standard | 8.5 | 8.2 | 9.4 | 7.2 | 6.0 |
| deviation | | | | | |
| CV (%) | 16.2 | 16.1 | 13.6 | 7.7 | 6.6 |

Recovery yields of between 50 and 95% were obtained.

5. Conclusion

It is determined that the isolation by magnetization of biotinylated latex beads made magnetic by the binding of FF-SA is feasible (sufficient recovery yields).

This magnetic separation is compatible with the implementation of multiplex assaying (no overlapping of the various microsphere categories).

This example perfectly illustrates the flexibility of the system.

The separate use of multiplexing microspheres and of separation nanospheres broadens the field of availability of the microspheres having required qualities (size, autofluorescence, density, material, surface chemistry, etc.). In fact, non-magnetic latexes are very readily accessible in all the ranges of the abovementioned characteristics, whereas the range of magnetic microspheres is very limited (<1% of catalog references). The choice may mean, for example, that different suppliers are necessary in order to create a significant series of multiplexing families, or may mean that special (and therefore expensive) productions are required.

Conversely, with the system proposed, any multiplexing microsphere, within the very broad range of non-magnetic latexes, can be used.

### Example 6

### Example of a Model of a Kit According to the Invention

Reagent 1—Functionalized microspheres: mixture of biotinylated microspheres coated with Ab specific for the Ag to be assayed. Bead concentration: 2500 microspheres of each specificity/μl (table below).

| | Fluo- | | | |
|---|---|---|---|---|
| | res- | | | |
| | cence | | | |
| Diameter | at | | | |
| (μm) | 675 nm | Supplier | Reference | Trapping Ab |
| 15 | − | Polymer | PL-Microspheres | Anti-I. globigii |
| | | Labo- | Plain White | PAb |
| | | ratories | 15 μm | |
| 10 | − | Dynal | Dynospheres | Anti-I. pseudomallei |
| | | Particles | | PAb |
| 8 | − | Polymer | PL-Microspheres | Anti-Y. pestis PAb |
| | | Labo- | Plain White | |
| | | ratories | 8 μm | |
| 4.4 | +++ | Bang's | QuantumPlex #5 | Anti-Ricin PAb |
| 4.4 | ++ | Lab. | QuantumPlex #4 | Anti-SEB MAb1 |
| 4.4 | + | | QuantumPlex #3 | Anti-Ova PAb |

Reagent 2—Ferrofluids-Streptavidin: streptavidin, captivate ferrofluid conjugate (Molecular Probes, Ref. C-21476).

Reagent 3—Visualizing reagent: mixture of fluorescent conjugates specific for the Ag to be assayed (table below).

| | | Concentration |
|---|---|---|
| | | for use |
| Visualizing Ab | Conjugated fluorochrome | (μg/ml) |
| Anti-B. globigii PAb | R-Phycoerythrin (R-PE) | 25 |
| Anti-B. pseudomallei PAb | Fluorescein | 50 |
| | isothiocyanate (FITC) | |
| Anti-Y. pestis PAb | R-PE | 50 |
| Anti-Ricin PAb | FITC | 50 |
| Anti-SEB MAb2 | FITC | 50 |
| Anti-Ova PAb | FITC | 50 |

Reagent 4—Standards: concentrated mixture (concentration to be defined) of the 6 Ag to be assayed.

A series of dilutions of this reagent is to be prepared extemporaneously (dilution in Reagent 1). When treated under the same conditions as the sample to be analyzed, this range (number of points to be determined) makes it possible to quantify the Ag present in the sample.

Reagent 5—Dilution buffer: PBS buffer/0.1% Tween 20, pH=7.2.

Reagent 6—Washing buffer: to be determined (PBS/0.1% BSA/0.09% NaN₃ or PBS/0.1% Tween 20, or other).

Reagent 7—Neutralizing buffer: solution of d-biotin at 200 μg/ml in distilled water.

The d-biotin prevents aggregation of the various biotinylated microspheres during magnetization (microspheres/biotin-SA/FF/SA-biotin/microspheres aggregation avoided by neutralizing SA/FF/SA with biotin to give biotin-SA/FF/SA-biotin, which cannot perform any bridging between the various biotin-microspheres).

Material necessary not provided: Dynal MPC magnet.

### Example 7

### Model of an Operating Protocol for the Multiplex Assaying of 3 Bacteria and 3 Proteins

1. A standard range is prepared by mixing reagents 4 and 5 as indicated below

| Tube | | | | | |
|---|---|---|---|---|---|
| T0 | T1 | T2 | T3 | . . . | Tn |

Reagent 4 (μl)
Reagent 5 (μl)

| | |
|---|---|
| Concentration | Bacterial Ag |
| | (bacteria/ml) |
| | Protein Ag |
| | (ng/ml) |

2. 800 μl of the sample to be analyzed or 800 μl of standard are introduced into 1 ml tubes (tube T0 to Tn).

3. 20 μl of reagent 1 are added.

4. The tubes are vortexed.

5. The tubes are placed on a rotary shaker for 8 minutes (possibility of reducing this time to 5 minutes being studied).

6. 30 μl of reagent 2 are added.

7. The tubes are vortexed.

8. The tubes are placed on a rotary shaker for 5 minutes.

9. 100 μl of reagent 7 are introduced.

10. The tubes are vortexed.

11. Incubation is carried out for 1 minute at ambient temperature (this incubation of 1 minute is not necessarily required).

12. The tubes are placed on the magnet for 2 minutes 30 seconds (possibility of reducing this time to 2 minutes being studied).

13. The medium is removed.

14. 200 μl of reagent 3 are added.

15. The tubes are vortexed.

16. Incubation is carried out for 10 minutes at ambient temperature.

17. 600 μl of reagent 6 are added.

18. The tubes are placed on the magnet for 2 minutes 30 seconds (possibility of reducing this time to 2 minutes being studied).

19. The medium is removed.

20. 500 μl of reagent 6 are added.

21. Analysis is carried out by FCM.

### Example 8

### Detection and Assaying of a Bacterium on 15 μm Microspheres After Magnetic Isolation

Aim: To detect and determine the concentration of B. globigii on biotinylated 15 μm beads loaded with anti-B. globigii PAb.

Materials and protocol: Those corresponding to examples 6 and 7.

Samples analyzed: dilutions of B. globigii spores in reagent 1. Ag concentrations of 160 000, 80 000, 40 000, 20 000, 10 000 and 5000 spores/ml.

Results:

| | | MFI FL2 |
|---|---|---|
| | MFI FL2 | corrected |
| Spores/ml | (a.u.) | (a.u.) |
| 160 000 | 17.300 | 17.001 |
| 80 000 | 7.760 | 7.461 |
| 40 000 | 3.930 | 3.631 |
| 20 000 | 2.110 | 1.811 |
| 10 000 | 0.923 | 0.624 |
| 5000 | 0.463 | 0.164 |
| 0 | 0.299 | 0 |

See FIG. 9.

### Example 9

### Multiplex Assaying of Ovalbumin on Fluorescent 4.4 μm Microspheres After Magnetic Isolation

Aim: To detect and determine the concentration of ovalbumin on biotinylated fluorescent 4.4 μm beads loaded with anti-ovalbumin PAb.

Materials and protocol: The materials and the protocol used are described in examples 6 and 7.

Samples analyzed: dilutions of ovalbumin in reagent 1. Ovalbumin concentration of 1.6, 0.8, 0.4, 0.2, 0.1 and 0.05 ng/ml.

Results:

| Ovalbumin | MFI FL1 | MFI FL1 |
|---|---|---|
| (ng/ml) | (a.u.) | corrected (a.u.) |
| 1.60 | 4.940 | 4.76 |
| 0.80 | 2.670 | 2.49 |
| 0.40 | 1.400 | 1.22 |
| 0.20 | 0.893 | 0.72 |
| 0.10 | 0.483 | 0.31 |
| 0.05 | 0.269 | 0.09 |
| 0 | 0.177 | 0 |

### Example 10

### Multiplex Flow Cytometry Analysis Applied to Molecular Genetics for the Search for SNPs

OLA-type test

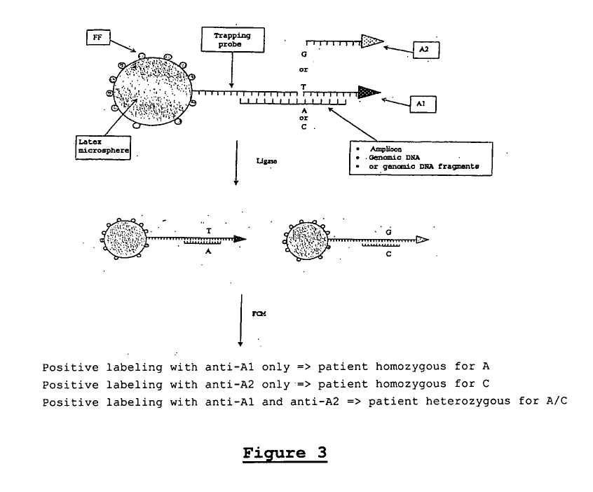

1) A trapping oligonucleotide probe, with generally between 5 and 100 bases in size, specific for a gene, is bound, by chemical methods, to a biotinylated latex microsphere (Iannone M A et al. 2000; Cytometry 39:131-140). The step consisting of biotinylation of the latex microsphere can also be carried out after the coupling of the trapping probe to the microsphere. The latex beads are incubated, in a single tube, in the presence of the streptavidin ferrofluids (FF-SA), of the visualizing probes (A1 and A2 in the example of FIG. 3) and of the amplicons or of the genomic DNA extracted from a biological sample not amplified beforehand by PCR or of the fragments derived from this DNA not amplified beforehand by PCR. The A1 and A2 probes can be either different fluorescent probes (for example, Cy3 and Cy5) or distinct haptens for a subsequent immunoreaction (for example, with two antibodies labeled with dissimilar fluorochromes [FITC versus PE]). The multiplexing is obtained by declination of the system of latex beads, which can either be variable in size and/or have distinct fluorescent characteristics.

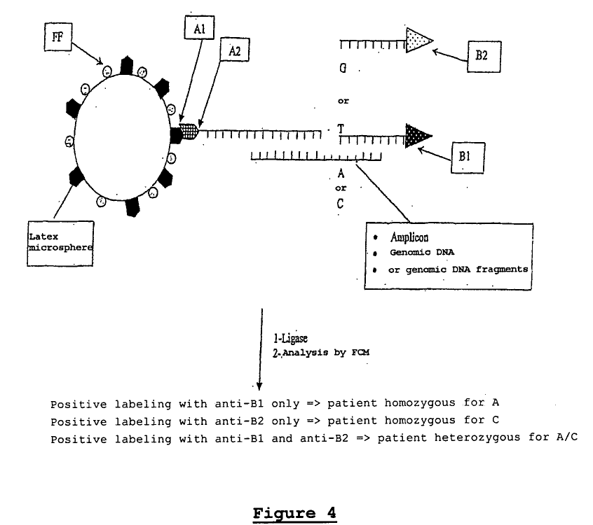

2) With the same principle as that stated in 1), a test can be developed by adding an additional degree of specificity in terms of the grafting of the trapping probe to the latex microsphere. This grafting can be carried out by means of a specific antigen-antibody couple, of a specific hapten-antibody couple or of a G+C (guanine, cytosine)-rich oligonucleotide probe. In this third case, a G+C-rich oligonucleotide sequence covalently bound to the microsphere hybridizes with a complementary sequence added in the 5′ position of the trapping probe (FIG. 4). In addition, in the case of the nucleotide hybridization, the Tm (melting temperature) of the anchoring nucleotide sequence will ideally be greater than 60° C. and/or composed of a polymer of at least 15 guanine or cytosine residues or of a mixture of the two nucleotide bases. Under these conditions, hybridizations and dehybridizations (generally carried out at between 15° C. and 40° C.) of the genomic material or of the amplicons trapped are possible without, however, dehybridizing the trapping probe from the microsphere.

### Example 11

### Differential Detection of Labeled PCR Fragments

1. Materials

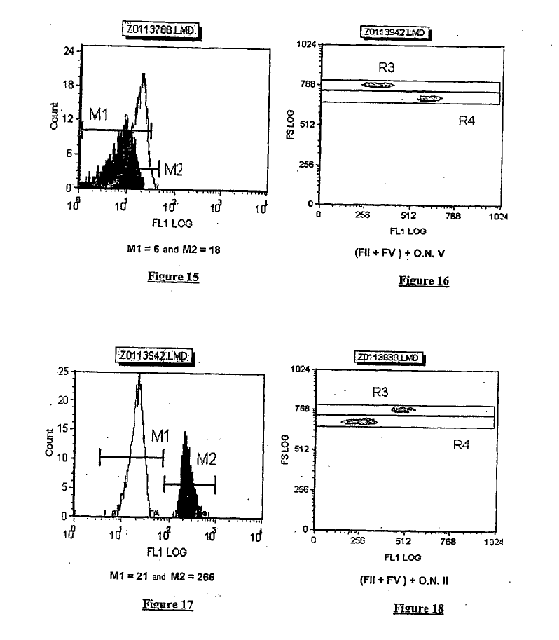

In certain approaches for carrying out the PCR, the PCR products are made fluorescent using labeled nucleotides. The technical approach proposed by the invention, which uses steps consisting in washing by magnetic separation of beads that are not initially magnetic, could apply to the differential detection of labeled PCR products, according to the principle shown diagrammatically in FIGS. 11 and 19 and which relates to 3 different specificities.

The feasibility of the Multiplex analysis by FCM after washing of the microspheres (US) with ferrofluids is illustrated in the example below and relates to 2 different specificities. The labeled PCR products are, in this case, modeled using oligonucleotides (O.N.) labeled with fluorescein, the sequences of which are complementary to those of the respective trapping probes, coupled beforehand to the surface of the beads. In practice:

the beads 6.7 μm in diameter (Sphero™ carboxyl-polystyrene particles, CP-60-10, Spherotech, Libertyville, Ill.) carry a factor V trapping probe constructed according to the structure below, indicated from the 5′ end to the 3′ end:
μS-FV: NH₂ (C₆) TTT TTT TTT TTT ggA cAA AAT Acc TgT ATT ccT c (SEQ ID No 1);

the beads 9.6 μm in diameter (PL-Microspheres SuperCarboxyl White 10 μm, Polymer Laboratories, UK) carry a factor II trapping probe constructed according to the structure below:
PS-FII: NH₂ (C₆) TTT TTT TTT TTT aat agc act ggg agc att gag gct c (SEQ ID No 2).

The 2 trapping probes are constructed with an amino group (—NH₂) in the 5′ position, with a view to covalent coupling to carboxylated beads (μS-COOH). They contain a brace arm (or spacer) made of up of 6 carbons (C₆) and 12 thymidines (T). All the oligonucleotides mentioned were synthesized specially by Proligo (Paris, F).

The biotin group at the surface of the beads, necessary for the system of the invention in the examples that follow, is introduced by means of a poly-(T)₃₀ oligonucleotide (referred to as polyT-biot), labeled in the 3′-position with biotin and carrying, in the 5′-position, an NH₂ brace (C₆). It is constructed according to the structure below:
NH₂ (C₆) TTT TTT TTT TTT TTT TTT TTT TTT TTT TTT-biotin (SEQ ID No 3)
and coupled in the same way and simultaneously with each trapping probe.

The oligonucleotides complementary to the trapping probes are constructed according to the structures below and are labeled with fluorescein (Fluor-) in the 5′ position:
for FV: Fluor-g Agg AAT AcA ggT ATT TTg Tcc (SEQ ID No. 4)
for FII: Fluor-g agc ctc aat gct ccc agt gct att (SEQ ID No. 5).

The coupling of the trapping probes to the caboxylated beads of corresponding diameter is carried out after activation with EDAC (N-(dimethylaminopropyl)-N′-ethylcarbodiimide HCl, Sigma) as follows:

For each type of bead, 10 million beads are washed in PBS buffer by centrifugation and adjusted to a concentration of 5×10⁶ μS/ml. The beads are activated by adding 0.8 mg of EDAC (80 μl at 10 mg/ml) and incubating for 30 min. The probes are subsequently brought into contact with the activated beads. In order to simultaneously carry out the coupling of the specific probe and of the biotin-carrying probe, an equimolar mixture of trapping oligonucleotide and of polyT-biot probe is brought into contact with the activated beads (at a final concentration of 17 nmol/ml, i.e. ˜250 μmol of each O.N. in total).

The beads are incubated with intermittent agitation (vortex) for 2 hours at AT (ambient temperature) in a glass tube. After the coupling, the activated carboxyl groups are neutralized by adding 400 μl of 0.2M ethanolamine and incubating for 16 h at 4° C.

Finally, the hydrophobic interaction sites of the beads are also saturated by incubation for 30 min, with agitation, in PBS-2% BSA.

For the tests of hybridization of the O.N. to beads described hereinafter, the buffers are the same as those used in the following example (example 12), where the detection effectively concerns double-stranded DNAs. These astringent or nonastringent, optimum-pH buffers, that allow, respectively, i) dehybridization of paired amplicons and ii) neutralization under conditions favorable to at least partial rehybridization (§) (cf. note of part 2 (materials) of example 12) to the immobilized trapping probes, are all derived from the Genecolor™ FV Leiden kit (BioCytex, Marseilles, F), under the respective names “hybridization buffer”/ and “ligation buffer”/ here referred to as neutralizing buffer.

The washing/dilution buffer for flow cytometry analysis is PBS-0.1% Tween 20®

(§) cf. note in the following example (cf. note of part 2 (materials) of example 12).

2. Methods

The beads (5 μl test, i.e. 100 000 μS/test for each type) and the complementary oligonucleotide (O.N.) (5 μl, i.e. 3 μmol/test for each of the O.N. for the maximum doses or dilution to 1/10 according to indications) are incubated in PCR tubes (Simport, Quebec, C) for 15 min at AT in hybridization buffer, and then for 15 min at AT in neutralizing buffer, so as to obtain hybridization of the complementary strands. After hybridization, the O.N. not bound to the beads are washed away by magnetic separation. For this, 10 μl of Captivate™, ferrofluids loaded with streptavidin (referred to as SA-FF, Molecular Probes, Eugene, Oreg., USA) are added to the reaction mixture, the mixture is incubated for 10 min, the content of the PCR tube is transferred into a tube for FCM, 1 ml of washing buffer (PBS-0.1% Tween 20®) is added and the tubes are placed against a powerful magnet (MPC-L, Dynal F, Compiègne, F) for 5 min. The magnetized beads remaining stuck against the tube wall, the liquid phase is removed, and the beads are resuspended in 2 ml of washing buffer for the next phase (selective dehybridization).

For the selective dehybridization, the tubes are heated to close to the melting point (Tm) of the probes so as to maintain only the specific hybridizations (corresponding to complete sequence complementarity, i.e. 100%) and to dissociate the nonspecific hybridizations (corresponding to partial sequence complementarities, i.e. <35%). For this, the tubes are incubated at 54° C. in PBS buffer/0.1% Tween 20®, which condition allows selective detachment of the FV and FII fluorescent probes from their noncomplementary sequence, “FII” and “FV”, respectively. The tubes are kept in a water bath for 5 min at the temperature indicated, and then immediately analyzed by FMC.

For complete dehybridization, the tubes are heated well beyond the melting point (Tm), in practice for 10 min at 80° C.

3. Results

FIGS. 11 to 19 illustrate the differential detection of labeled oligonucleotide fragments representative of the FV and FII genes, respectively, in duplex cytometric analyses.

In all cases, the trapping beads with FV specificity (μS-FV) and 6.7 μm in diameter are pinpointed in the R1 region for analysis of their fluorescence. The trapping beads with FII specificity (μS-FII) and 9.6 μm in diameter are pinpointed in the R2 region.

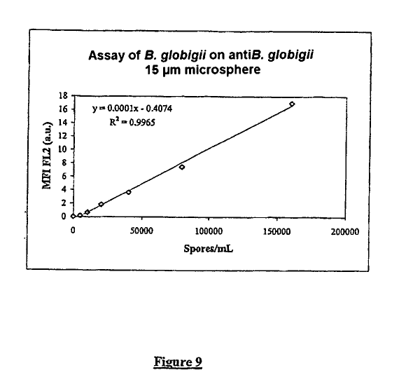

a) In the presence of the two labeled oligonucleotides simultaneously, maximum labeling of each type of bead is observed (FIGS. 12 and 13), corresponding to maximum mean intensities of, respectively:

831 arbitrary units (a.u.) for μS-FV

247 arbitrary units (a.u.) for μS-FII

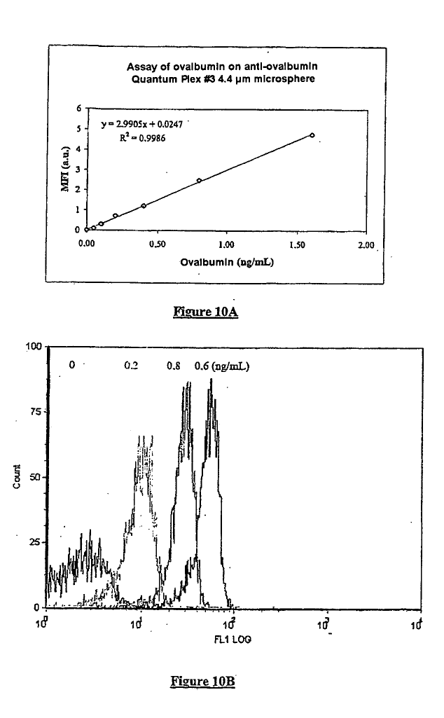

b) When the same beads are subjected to complete dehybridization by heating for 10 min at 80° C. (FIGS. 14 and 15), minimum labeling of each type of bead is observed (FIGS. 14 and 15), corresponding to minimum mean intensities of:

6 a.u. for μS-FV

18 a.u. for μS-FII.

These results therefore correspond to 2 working ranges of maximum amplitudes of, respectively:

6 to 840 a.u. (μS-FV)

18 to 250 a.u. (μS-FII).

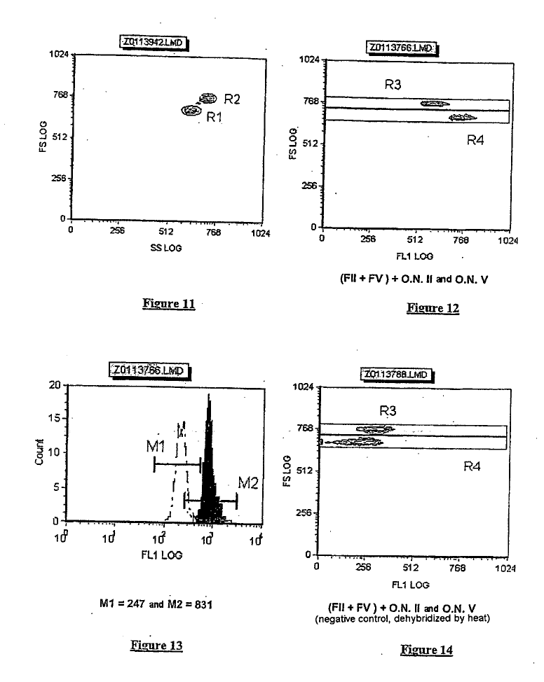

c) In the presence of a reduced dose (1/10 of the maxi dose) of just one of the 2 labeled oligonucleotides (FV), strong labeling is observed on μS-FV (FIGS. 16 and 17; 266 a.u., i.e. ˜25% of the maximum possible amplitude) whereas, for PS-FII, the signal remains close to the BN of 18 a.u. seen in FIG. 15 (21 a.u., i.e. <2% of the maximum possible amplitude).

d) Conversely, in the presence of a reduced dose (1/10 of the maxi dose) of just one of the 2 labeled oligonucleotides (FII), positive labeling is observed on μS-FII (77 a.u., i.e. ˜30% of the maximum possible amplitude) whereas, for PS-FV, the signal remains weak (11 a.u., i.e. <2% of the maximum possible amplitude) but nevertheless greater than the BN of 6 a.u. seen in FIG. 15, which suggests the existence of a slight residual nonspecific labeling.

The table below summarizes the results regarding the duplex FCM analysis of the O.N. representative of the factor II and factor V genes and shows that the detection of fluorescence-labeled DNA fragments is simple and can be carried out in a single tube.

| | | Trapping μS | | | |
|---|---|---|---|---|---|
| | | | μS-FV | | μS-FII |
| | | | MFI | | MFI |
| Alleles present | FIGS. | μS-FV | (a.u.) | μS-FII | (a.u.) |
| FV + FII | 12 and 13 | Positive | 831 | Positive | 247 |
| Heating >> Tm | 14 and 15 | Negative | 6 | Negative | 18 |
| (neg. control) | | | | | |
| FV | 16 and 17 | Positive | 266 | Negative | 21 |
| FII | 18 and 19 | Negative | 11 | Positive | 77 |

### Example 12

### Differential Detection of an SNP Mutation

1. Principle

The detection of point mutations (SNPs) is based on the OLA technique (Landegren, Science 1988, 241: 1077-80). This technique involves:

The formation of a ternary complex between the trapping probe (immobilized in this case on a family of beads), the complementary single-stranded DNA strand derived from the amplicons of the corresponding gene and a visualizing probe contiguous to the trapping probe.

The covalent coupling of the trapping probe and of the visualizing probe—hydridized with the complementary strand—by the specific action of a ligase that joins together only strands that are exactly contiguous and perfectly hybridized. The lack of complementarity on the sole base carrying the mutation is sufficient to prevent this coupling (here referred to as ligation).

Dissociation, under astringent conditions, of the DNA double strands.

When the ligase finds itself under the specificity conditions required for its action, and only in this case, the visualizing probe remains associated (covalent coupling) with the trapping probe and therefore with the corresponding support (in this case a bead).

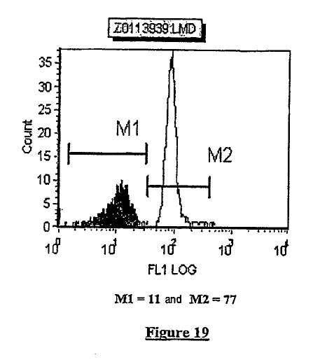

The application of this principle in the context of the invention is illustrated by FIGS. 20 and 21.

Comment: FMC makes it possible to simultaneously measure fluorescence intensities of very different levels (from background noise to ++++ labeling) on groups of beads that can be differentiated on the basis of another parameter (size or fluorescence of different wavelengths).

2. Materials

The detection of point mutations (SNPs), the principle of which is illustrated by FIG. 21, requires the use of two types of μS for differential detection. The μS used here are loaded with the trapping probes ad hoc as illustrated in example 11 and are such that:

the beads 6.7 μm in diameter carry a wild-type factor V trapping probe (μS-FVwt) constructed according to the structure below, indicated from the 5′ to the 3′ end:
μS-FVwt: NH₂ (C₆) TTT TTT TTT TTT ggA cAA AAT Acc TgT ATT ccT c (SEQ ID No. 1);

the beads 9.6 μm in diameter carry a mutant factor V trapping probe (μS-FVmut) constructed according to the structure below, indicated from the 5′ to the 3′ end:
μS-FVmut: NH₂ (C₆) TTT TTT TTT TTT ggA cAA AAT Acc TgT ATT ccT T (SEQ ID No. 6).

The biotin group at the surface of the beads, required for the system of the invention in the examples that follow, is introduced as in paragraph A by means of a poly-(T)₃₀ oligonucleotide and allows specific binding of streptavidin-ferrofluid (Captivate™, Molecular Probes, Eugene, Oreg., USA).

The probe contiguous with the trapping probe that allows visualization of the ligation carries a phosphate group in the 5′-position and fluorescein labeling in the 3′-position; it was specially synthesized by Proligo (Paris, F) and has the following sequence:
PO₄²⁻-gcc TgT ccA ggg ATc TgcTcc-fluo (SEQ ID No. 7).

The amplicons for the formation of the ternary complex are derived from the PCR amplification of a fragment of the wild-type and/or mutant factor V gene from genomic DNA or from specific plasmids, which PCR is carried out in the presence of an “Ampli-Mix” reagent PCR mix available in the Genecolor™ FV Leiden kit (BioCytex, Marseilles, F).

The ligase solution for the formation of a covalent bond between the trapping probe and the signal probe is the “ligation solution” reagent, i.e. a T4 ligase in its special “ligation buffer”, as used in the Genecolor™ FV Leiden kits (Biocytex, Marseilles, F).

The buffers used hereinafter are of optimal pH, are astrigent or nonastringent, and allow, respectively:

i) dehybridization of the paired amplicons and partial rehybridization thereof (§) (cf. note of part 2 (materials) of example 12) to the immobilized trapping probes,

ii) the action of the ligase, and finally,

iii) dehybridization of the amplicons and probes not coupled after ligation.

They are also all derived from the Genecolor™ FV Leiden kit, under the respective names “hybridization buffer”/“ligation buffer” (also referred to as neutralizing buffer in example 11) and “washing buffer”.

The dilution buffer for flow cytometry analysis is PBS-0.1% Tween 20®.

(§) The stoichiometric conditions are optimized beforehand (excess of amplicons, excess of signal probe) so as to obtain rehybridization of a significant fraction of one of the 2 DNA strands to the immobilized trapping probes, rather than to its complementary strand.

3. Protocol

The 2 types of beads (μS-FVwt and μS-FVmut) are mixed in equivalent amounts and diluted in hybridization buffer in a proportion of 40 000 μS/μl in total. 5 μl of suspension of beads (i.e. 100 000 μS/test for each type), the amplicons (3.75 μl/test) and the FV visualizing probe (1 μmol in 1.25 μl of hybridization buffer) are distributed into a special PCR microtube (PCR T 320-1N, Simport, Quebec, C). The reaction medium is homogenized (vortex) and incubated for 30 min at ambient temperature (AT).

The ligation step is subsequently carried out by incubation for one hour after the addition of 100 μl of ligation solution.

After ligation, the excess amplicons and excess signal probe not involved in the ternary complex are removed by magnetic separation. For this, the reaction mixture (Vt=110 μl) has 10 μl of ferrofluid suspension (SA-FF) added to it. After agitation (vortex) and incubation for 10 min, the mixture is transferred into a 1 ml tube (Ringer tubes) and the magnetization is carried out for 5 min with a powerful magnet (MPC-S, Dynal, Compiegne, F). The beads, which are stuck against the wall of the tube, are dried off by aspiration of the liquid and resuspended with 100 μl of washing solution. The washing solution, having suitable characteristics, allows selected detachment of the products associated with the beads only by noncovalent interaction (hybridization without ligation), but not that of the probes covalently bound nor that of the SA-FF. This washing is repeated a second time.

The beads are finally diluted in 1 ml of dilution buffer (PBS-Tween 20®), transferred into a tube for cytometry (4 ml) and analyzed by FCM.

4. Results

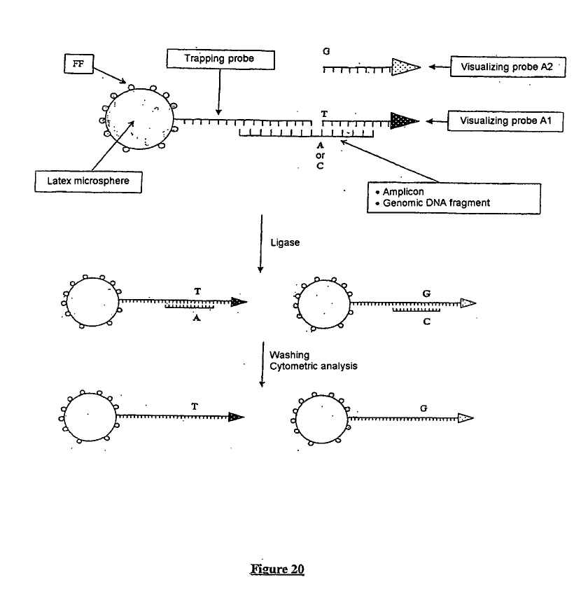

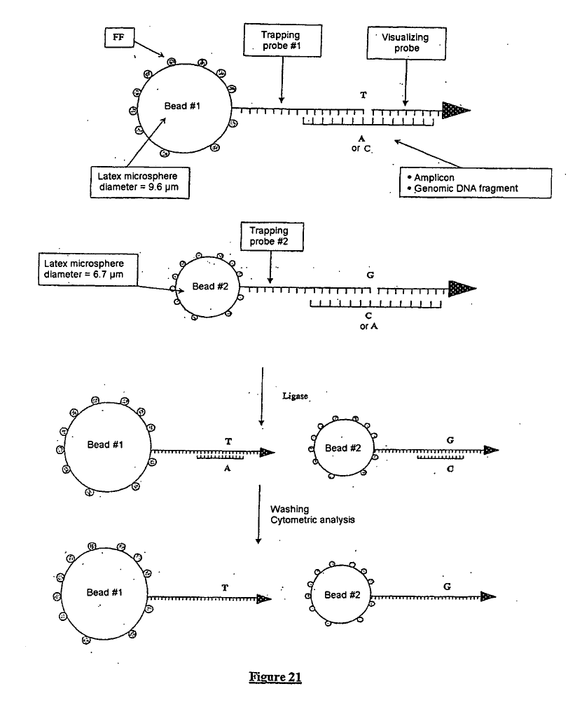

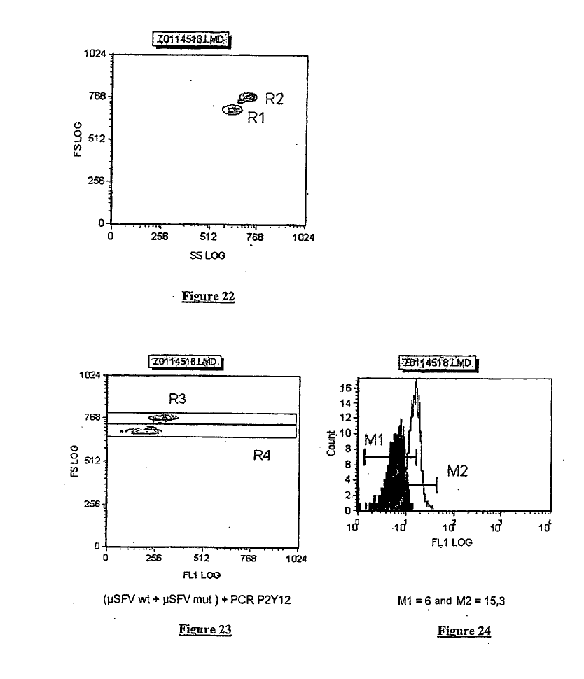

a) In the presence of PCR products not recognizable by the system (in this case, products derived from the amplification of the P2Y12 gene, wild-type allele, generated with the reagents of the Genecolor™ P2Y12 G52T kit, BioCytex, Marseilles, F), each of the 2 beads gives labeling similar to its intrinsic background noise (BN), in practice (FIGS. 23 and 24);

μS-FVwt: 6.0 a.u.

μS-FVmut: 15.3 a.u.

b) In the presence of PCR products corresponding to the wild-type allele of the factor V gene (FVwt, PCR generated with the reagents of the Genecolor™ factor V Leiden kit, BioCytex, Marseilles, F), the μS-FVwt beads show a clearly positive labeling whereas the μS-FVmut beads give labeling similar to their intrinsic background noise, in practice (FIGS. 25 to 28):

μS-FVwt: 313 a.u. (versus BN at 6.0 a.u.)

μS-FVmut: 17.4 a.u. (versus BN at 15.3 a.u.).

The signal of each bead in the test is superposed on its nonspecific BN (μS-FVwt: FIG. 27; μS-FVmut: FIG. 28). This suggests a broad working range for the FVwt specificity (from 6 to more than 300 a.u.) and virtually zero nonspecific labeling on the FVmut bead, in the absence of its specific ligand (FVmut amplicons).

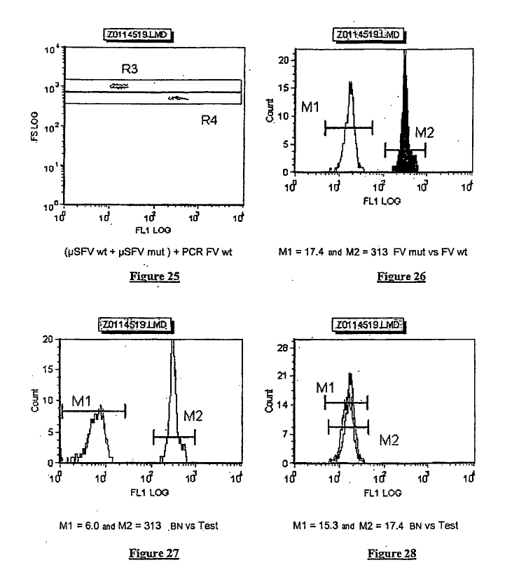

c) In the presence of PCR products corresponding to the mutated allele of the factor V gene (FVmut, PCR generated with the reagents of the Genecolor™ FV Leiden kit, BioCytex, Marseilles, F), the μS-FVmut beads show labeling that is clearly different from the BN, corresponding to the maximum positive signal level possible with the material available. The μS-FVwt beads give weak labeling compared with the maximum positive signal (14.8 versus 313 a.u., i.e. <2% of the maximum amplitude of variation), although it is different from their intrinsic background noise, which suggests the existence of a weak but real nonspecific labeling on these beads in the presence of FVmut amplicons. In practice (FIGS. 29 to 32):

μS-FVwt: 14.8 a.u. (versus BN at 6.0 a.u.)

μS-FVmut: 43.4 a.u. (versus BN at 15.3 a.u.).

For the detection of this FV mutation, optionally in the presence of the other allele, the respective working amplitudes (ranges) are therefore, at best:

FVwt: from 15 to 300 a.u.

FVmut: from 17 to 43 a.u.

The shift observed with respect to the maximum intensity (300 a.u. versus 43 a.u.) can be attributed to a poorer coating efficiency for the μS-FVmut beads; as in Example No. 11, these 9.7 μm beads give a poorer level of coating. It should be noted that, by virtue of the principle of the invention, other bead batches, types, origins and diameters can be used at will in order to obtain the optimal characteristics of load capacity and/or of intrinsic BN, without worrying about their magnetic properties, which significantly extends the choice of supply.

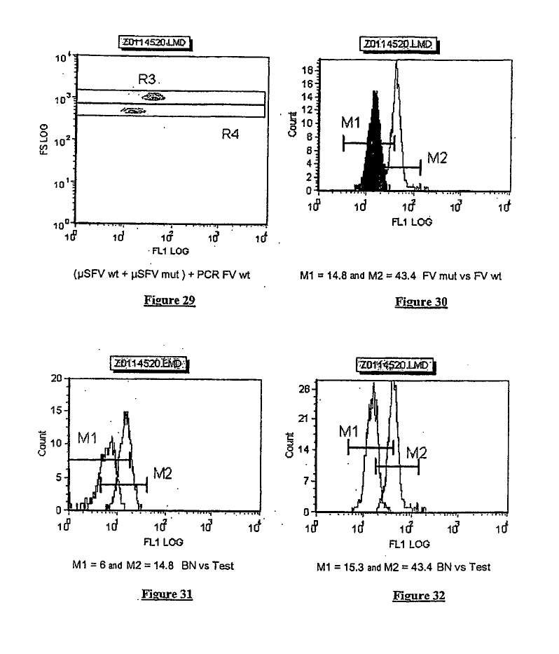

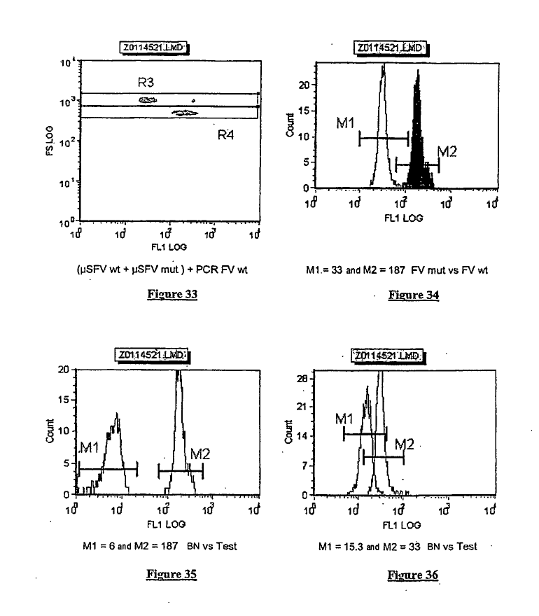

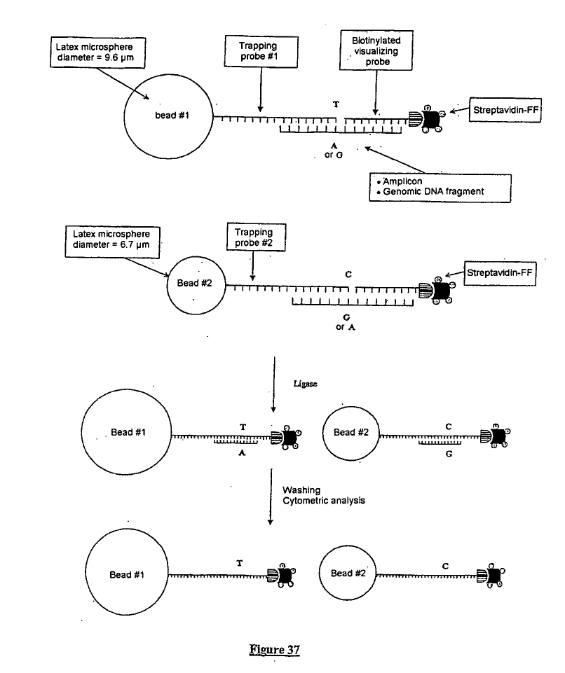

d) In the presence of PCR products corresponding to the 2 alleles of the factor V gene simultaneously (FVmut and FVwt, PCR generated with the reagents of the Genecolor™ FV Leiden kit, BioCytex, Marseilles, F), the μS-FVmut beads and the μS-FVwt beads show a clearly positive labeling, although at weaker intensity levels than those observed with the amplicons specific for a single allele, used alone, in practice (FIG. 33 to 36):

μS-FVwt: 187 a.u. (i.e. ⅔ of the maximum specific labeling amplitude: [187-15]/[300/15])

μS-FVmut: 33 a.u. (i.e. ⅔ of the maximum specific labeling amplitude: [33-17]/[43/17]).

The table below summarizes the results on the duplex FCM analysis of the Leiden mutation of factor V, and shows that the definition of the FV genotype is simple and can be carried out in a single tube.

| | | Trapping μS | | | |
|---|---|---|---|---|---|
| | | | μS-FV | | μS-FV |
| | | | wt | | mut |
| Alleles | | μS-FV | MFI | μS-FV | MFI |
| present | FIGS. | wt | (a.u.) | mut | (a.u.) |
| P2Y12G52 | 23 to 24 | Negative | 6.0 | Negative | 15.3 |
| (neg. control) | | | | | |
| FV wt/wt | 25 to 28 | Positive | 313 | Negative | 17.3 |
| FV mut/mut | 29 to 32 | Negative | 14.8 | Positive | 43.4 |
| FV wt/mut | 33 to 36 | Positive | 187 | Positive | 33 |

5. Extensions

The above examples, in particular Nos. 1, 2 and 5, have already illustrated, in the context of immunological detections, the possibility of using a larger number of families of beads that can be readily differentiated through their sizes and/or a variable level of a second fluorescence that is different from that used for the measurement. It emerges from the agreement of all these examples that a Multiplex analysis of the 2 alleles of the FV and FII genes in a single tube would be very easy, using, for example:

the same carboxylated beads as illustrated in example 12 (FVmut diameter 9.6 μm and FVwt diameter 6.7 μm) and also the carboxylated beads of 4.4 μm and carrying two different levels of red fluorescence as illustrated in examples 4 and 5, for carrying the two probes specific for the wild-type and mutant alleles of FII.

Two different fluorescences for the measurement according to the principle of FIG. 20, which requires just one type of bead per mutation.

These two approaches can be generalized by considering that:

the first, using only one fluorescence for the measurement, makes it possible to detect as many different alleles as the number of bead families that can be differentiated simultaneously,

the second, using two different fluorescences for the measurement, makes it possible to genotype as many genes as the number of bead families that can be differentiated simultaneously.

In all cases, the major advantage provided by the invention is that the choice of beads for the multiplex analysis does not impose any limiting condition on their intrinsic magnetic properties.

### Example 13

### Differential Detection of an SNP Mutation

1. Principle of FIG. 37:

In FIG. 37, as in FIG. 21, the critical base (SNP specificity) is carried by each of the allele-specific trapping probes, each coupled to a different type of bead (differentiated by size). This system calls, for the signal analysis by FCM, for only an analysis by counting the beads on the basis of the size and structure parameters, making it possible either to use a device that does not have a fluorescence detector and is therefore less expensive, or to take advantage of other sizes for differentiating the bead families with respect to one another.

2. Materials:

The detection of point mutations (SNPs), the principle of which is illustrated in FIG. 37, requires the use of two types of μS for differential detection. The μS used here are loaded with the trapping probes ad hoc as illustrated in paragraph A and are such that:

the beads 6.7 μm in diameter carry a wild-type factor V trapping probe (μS-FVwt) constructed according to the structure below, indicated from the 5′ end to the 3′ end:
μS-FVwt: NH₂ (C₆) TTT TTT TTT TTT ggA cAA AAT Acc TgT ATT ccT c (SEQ ID No 1);

the beads 9.6 μm in diameter carry a mutant factor V trapping probe (μS-FVmut) constructed according to the structure below, indicated from the 5′ to the 3′ end:
μS-FVmut: NH₂ (C₆) TTT TTT TTT TTT ggA cAA AAT Acc TgT ATT ccT T (SEQ ID No. 6).

The probe contiguous to the trapping probe, that makes it possible to visualize the ligation, carries a 5′-phosphate group and a 3′-biotin group; it was specially synthesized by Proligo (Paris, F) and has the following sequence:
PO₄²⁻-gcc, TgT ccA ggg ATc TgcTcc TTT TTT TTT TTT TTT TTT-Biotin (SEQ ID No. 8).

The biotin group on the trapping probe, necessary for the system of the invention in the examples that follow, allows the specific binding of ferrofluid-streptavidin (Captivate™, Molecular Probes, Eugene, Oreg., USA). The amplicons that allow the formation of the ternary complex are derived from the PCR amplification of a fragment of the wild-type and/or mutant factor V gene from genomic DNA or from specific plasmids, which PCR is carried out in the presence of an “Ampli-Mix” reagent PCR mix available in the Genecolor™ FV Leiden kit (BioCytex, Marseilles, F).

The ligase solution for the formation of a covalent bond between the trapping probe and the signal probe is the “ligation solution” reagent, i.e. a T4 ligase in its special “ligation buffer”, as used in the Genecolor™ FV Leiden kit (Biocytex, Marseilles, F).

The buffers used hereinafter are of optimal pH, are astringent or nonastringent and allow, respectively,

i) dehybridization of the paired amplicons and partial rehybridization thereof (§) (cf. below) with the immobolized trapping probes,

ii) the action of the ligase and, finally

iii) dehybridization of the amplicons and probes not coupled after ligation.

They are also all derived from the Genecolor™ FV Leiden kit, under the respective names “hybridization buffer”, “ligation buffer” (also referred to as neutralizing buffer in example A) and “washing solution”.

The dilution buffer for flow cytometry analysis is PBS-0.1% Tween 20®.

(§) The stoichiometric conditions are optimized beforehand (excess of amplicons, excess of signal probe) so as to obtain the rehybridization of a significant fraction of one of the 2 DNA strands with the immobilized trapping probes rather than with its complementary strand.

3. Protocol:

The two types of beads (μS-FVwt and μS-FVmut) are mixed in equivalent amounts and diluted in hybridization buffer in a proportion of 40 000 μS/μl in total. 5 μl of bead suspension (i.e. 100 000 μS/test for each type), the amplicons (3.75 μl/test) and the FV visualizing probe (1 μmol in 1.25 μl of hybridization buffer) are distributed into a special PCR microtube (PCR T 320-1N, Simport, Quebec, C). The reaction medium is homogenized (vortex) and incubated for 30 min at ambient temperature (AT).

The ligation step is subsequently performed by incubation for one hour after the addition of 100 μl of ligation solution.

After ligation, the excess of amplicons and of signal probe not involved in the ternary complex is removed by magnetic separation. For this, the reaction mixture (Vt=110 μl) has 10 μl of ferrofluid suspension (SA-FF) added to it. After agitation (vortex) and incubation for 10 min, the mixture is transferred into a 1 ml tube (Ringer tubes) and the magnetization is carried out for 5 min with a powerful magnet (MPC-S, Dynal, Compiegne, F). The beads, stuck against the tube wall, are dried out by aspiration of the liquid and resuspended with 100 μl of washing solution. The washing solution, having suitable characteristics, allows selective detachment of the products associated with the beads only by noncovalent interaction (hybridization without ligation), but not that of the covalently bound probes nor that of the SA-FF. This washing is repeated a second time.

The beads are finally diluted in 1 ml of dilution buffer (PBS-Tween 20®), transferred into a tube for cytometry (4 ml) and analyzed by FCM.

4. Results:

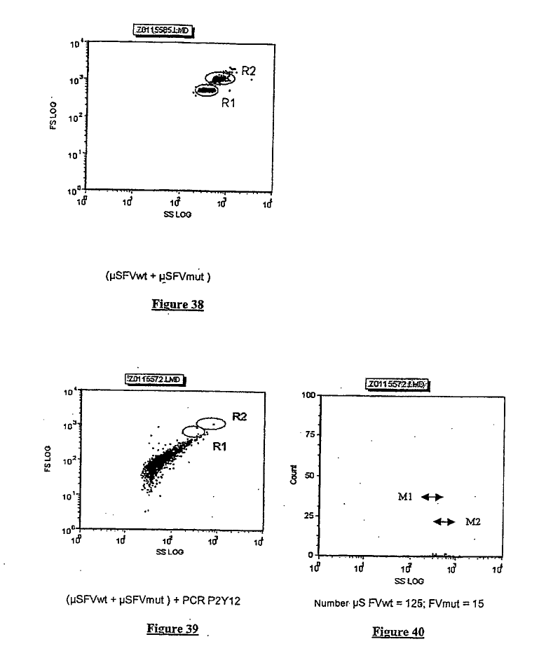

a) In the presence of PCR products not recognizable by the system (in this case, products derived from the amplification of the P2Y12 gene, wild-type allele, generated with the reagents of the Genecolor™ P2Y12 G52T kit, BioCytex, Marseilles, F), each of the 2 beads gives labeling similar to its intrinsic background noise (BN), in practice (FIG. 40):

number of μS-FVwt: 125

number of μS-FVmut: 15.

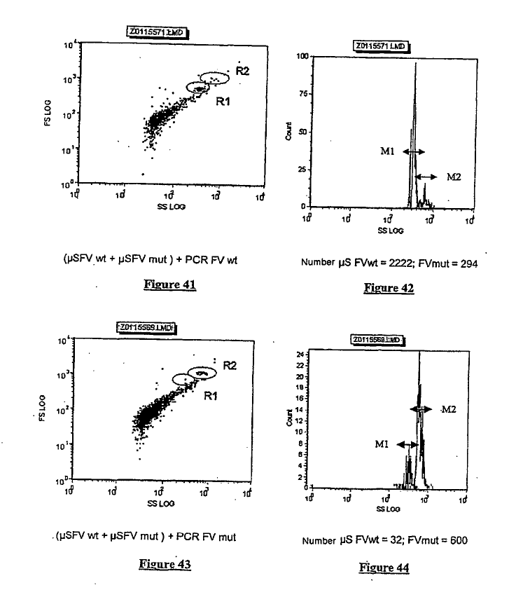

b) In the presence of PCR products corresponding to the wild-type allele of the factor V gene (FVwt, PCR generated with the reagents of the Genecolor™ factor V Leiden kit, BioCytex, Marseilles, F), the μS-FVwt beads show a clearly positive labeling whereas the μS-FVmut beads give labeling similar to their intrinsic background noise, in practice (FIGS. 41 and 42):

number of μS-FVwt: 2222

number of μS-FVmut: 294.

c) In the presence of PCR products corresponding to the mutated allele of the factor V gene (FVmut, PCR generated with the reagents of the Genecolor™ FV Leiden kit, BioCytex, Marseilles, F), the μS-FVmut beads show labeling that is clearly different from the BN, corresponding to the maximum level of positive signal possible with the positive available material, whereas the μS-FVwt beads give labeling similar to their intrinsic background noise. In practice (FIGS. 43 and 44):

number of μS-FVwt: 32

number of μS-FVmut: 600.

The table below summarizes the results on the duplex FCM analysis of the Leiden mutation of factor V, and shows that the definition of the FV genotype is simple and can be carried out in a single tube.

| | | Trapping μS | | | |
|---|---|---|---|---|---|
| | | μS-FV wt | | μS-FV mut | |
| | | (6.7 μm) | | (9.6 μm) | |
| | | | Number | | Number |
| Alleles | | | of | | of |
| present | FIGS. | | beads | | beads |
| P2Y12G52 | 39 and 40 | Negative | 125 | Negative | 15 |
| (neg. control) | | | | | |
| FV wt/wt | 41 and 42 | Positive | 2222 | Negative | 294 |
| FV mut/mut | 43 and 44 | Negative | 35 | Positive | 600 |

1. Method for the detection and/or multiplex quantification of analytes that may be contained in a sample, using functionalized non-magnetic microspheres, it being possible, where appropriate, for said analytes to be labeled beforehand with a label, said method being characterized in that it comprises the following steps:
a) bringing said sample into contact with a suspension of functionalized non-magnetic microsphere populations, said microspheres carrying at their surface:
for all the microsphere populations, a compound A forming a first member of a binding pair, said compound A also being characterized in that it cannot bind with said analytes, and
for each one of the microsphere populations, a compound B, that is different for each population, capable of forming a specific binding pair with one of said analytes of the sample,
b) adding to the reaction medium obtained in step a) a ferrofluid, which ferrofluid contains magnetic particles which carry at their surface a second binding member capable of forming a specific binding pair with the compound A,
c) at least one step consisting in washing by magnetic separation of the microspheres magnetized in step b),
d) where appropriate, when said analytes are not labeled beforehand, bringing the suspension of magnetized microspheres obtained in step c) into contact with a solution of at least one conjugate, said conjugate comprising a compound C capable of recognizing and of binding specifically with one of said analytes and a label, this step
d) preferably being followed by at least one step consisting in washing the microspheres by magnetic separation, and
e) detecting and/or quantifying said label at the surface of the microspheres.

2. The method of claim 1, characterized in that at least two of said microsphere populations also have at least one intrinsic physical characteristic that makes it possible to differentiate them from one another.

3. The method of claim 1, characterized in that said binding pair formed between the compound A and the second member bound to the surface of the ferrofluids is preferably chosen from the group consisting of the specific binding pairs of biotin/avidin or biotin/streptavidin, enzyme/cofactor, lectin/carbohydrate and antibody/hapten type.

4. The method of claim 1, characterized in that the microspheres are made of a material chosen from the group consisting of latex, a polymer, a copolymer, glass and silica.

5. The method of claim 1, characterized in that the label(s) is (are) fluorescent.

6. The method of claim 1, characterized in that the detection and/or the quantification of said label in step e) of the method is carried out by flow cytometry.

7. The method of claim 6, characterized in that said intrinsic physical characteristic that makes it possible to differentiate the at least 2 microsphere populations is the size and/or an optical property of said microspheres.

8. The method of claim 1, characterized in that the microspheres have a size of between 0.3 and 100 μm in diameter.

9. The method of claim 7, characterized in that the optical property is the emission wavelength and/or the fluorescence intensity of said microspheres.

10. The method of claim 1, characterized in that said analytes are of protein and derivatives type, or are nucleic acids.

11. The method of claim 1, characterized in that said analytes are compounds that may be either present in solution in a liquid, or present at the surface of a cell or of a particle in suspension in the sample.

12. The method of claim 1, characterized in that said compounds are protein toxins, or in that said cell or particle is a microorganism, such as a bacterium or a virus.

13. The method of claim 1, characterized in that said compound B is chosen from the group consisting of proteins and nucleic acids.

14. The method of claim 1, characterized in that said compound C of said conjugate used in step d) is chosen from the group consisting of proteins and nucleic acids.

15. The method of claim 1, characterized in that said analytes are nucleic acids and in that, in step a), the compound B is a nucleic acid capable of hybridizing specifically with one of said analytes.

16. The method of claim 15, characterized in that said analytes are PCR products.

17. The method of claim 16, characterized in that said PCR products are obtained labeled.

18. The method of claim 15, characterized in that said compound C of said conjugate is a nucleic acid capable of hybridizing specifically with one of said analytes.

19. The method of claim 18, for the detection and/or multiplex quantification of SNPs.

20. The method of claim 19, characterized in that the detection and/or multiplex quantification of SNPs is carried out by the OLA method.

21. Kit for the detection and/or multiplex quantification of analytes that may be contained in a sample, characterized in that it comprises:
a) a reagent 1 comprising a suspension of populations of functionalized non-magnetic microspheres, said microspheres carrying at their surface:
a compound A forming a first member of a binding pair;
a compound B capable of forming a specific binding with one of said analytes of the sample, and
b) a reagent 2 comprising a ferrofluid which contains magnetic particles carrying at their surface a second binding member capable of forming a specific binding pair with said compound A; and
c) a reagent 3 comprising a solution of at least one conjugate, said conjugate comprising a compound C capable of reacting specifically with said analytes, and a label capable of being detected.

22. The kit of claim 21, characterized in that it also comprises:
a reagent 4 comprising said analytes that may be contained in a sample.

23. The kit of claim 21, characterized in that it also comprises:
a reagent 5 composed of a dilution buffer; and
a reagent 6 composed of a washing buffer.

24. The kit of claim 21, characterized in that it also comprises:
a reagent 7 comprising a buffer for neutralizing the aggregation of the various microspheres.
